(19) United States
(12) Patent Application Publication
(10) Pub. No.: US 2026/0199498 A1
(43) Pub. Date: Jul. 16, 2026

(54) CANCER TREATMENT WITH AN RORI ANTIBODY IMMUNOCONJUGATE AND A BTK INHIBITOR

(71) Applicant: Merck Sharp & Dohme LLC, Rahway, NJ (US)

(72) Inventors: Uzor C. Ogbu, Media, PA (US); Nishitha Reddy, Nolensville, TN (US); Zifang Guo, Princeton, NJ (US); Mohammed Z.H. Farooqui, Gwynedd, PA (US); Patricia Marinello, Philadelphia, PA (US); Ying Zhu, Weehawken, NJ (US)

(73) Assignee: Merck Sharp & Dohme LLC, Rahway, NJ (US)

(21) Appl. No.: 19/133,519

(22) PCT Filed: Jan. 12, 2023
(86) PCT No.: PCT/US2023/010653

§ 371 Date: May 28, 2025

Related U.S. Application Data
(60) Provisional application No. 63/428,744, filed on Nov. 30, 2022.

(51) Int. Cl. A61K 47/68; A61K 31/519; A61P 35/00

(52) U.S. Cl. CPC A61K 47/68031; A61K 31/519; A61K 47/6849; A61K 47/6889; A61P 35/00

(57) ABSTRACT

Provided herein are methods for treating a cancer patient with an ROR1 immunoconjugate and a BTK inhibitor. These methods may be useful for the treatment of a variety of cancers that express ROR1 and are expected to be mediated or modulated by BTK signaling.

### CROSS REFERENCE TO RELATED APPLICATIONS

The present application claims the benefit of U.S. Provisional Application No. 63/428,744, filed Nov. 30, 2022, and is hereby incorporated by reference in its entirety.

### SEQUENCE LISTING

The instant application contains a Sequence Listing in XML format and is hereby incorporated by reference in its entirety. Said XML copy is named 25615-WO-PCT_SL.xml and is 8,192 bytes in size.

### BACKGROUND OF THE INVENTION

Receptor tyrosine kinase-like orphan receptor 1 (ROR1) is a cell-surface protein that mediates signals from its ligand, the secreted glycoprotein Wnt5a. Consistent with its role in influencing the fate of stem cells during embryogenesis, ROR1 expression is observed on invasive malignancies that revert to an embryonic transcriptional program. ROR1 is commonly expressed on the malignant cells of patients with hematological cancers, and is also present on the cell surfaces of multiple solid tumors, where it appears to be a marker for cancer stem cells. Nevertheless, ROR1 expression is not commonly observed in normal adult tissues, and thus ROR1 presents a favorable selectivity profile as a therapeutic target.

Brunton's tyrosine kinase (BTK) is a non-receptor tyrosine kinase expressed in cells of all hematopoietic lineages except for T and plasma cells. BTK is a key regulator of the BCR signaling pathway. BTK regulates all aspects of B-cell development, including proliferation, maturation, differentiation, apoptosis, and cell migration. Since BTK plays a major role in the initiation, survival, and progression of B-cell lymphoproliferative malignancies, BTK inhibitors (BTKis) have been investigated as potential treatments of hematological cancers.

Mantle cell lymphoma (MCL) is an aggressive small B-cell lymphoma that develops in a linear fashion from naïve B cells. MCL comprises approximately 3% of non-Hodgkin lymphomas (NHLs), and generally has an aggressive course and a poor prognosis. Annual incidence is 0.5 to 1 case per 100,000 population and the rate of new cases has increased over recent years. Chromosomal t(11;14)(q13;q32) translocation is identified in >95% of all MCL cases, and is the primary oncogenic event in the pathogenesis of MCL, leading to overexpression of cyclin D1. Most patients start treatment at MCL diagnosis. However approximately 25% of patients have an indolent form that does not require immediate treatment. MCL remains incurable, with National Comprehensive Cancer Network (NCCN) guidelines recommending initial treatment based on patient fitness. For fit patients <65 years old, NCCN recommends aggressive treatment that consists of high-dose chemoimmunotherapy in combination with rituximab followed by consolidation with high-dose therapy and autologous stem cell transplant (ASCT). The addition of rituximab maintenance improves overall survival after ASCT. Less aggressive treatments for frail and older patients include bendamustine and rituximab (BR); rituximab, cyclophosphamide, doxorubicin, vincristine, and prednisone (R-CHOP); bortezomib plus rituximab, cyclophosphamide, doxorubicin, and prednisone (VR-CAP); lenalidomide and rituximab (R²); and rituximab, bendamustine, and cytarabine (R-BAC500). Despite high initial response rates, most patients eventually relapse.

The anti-ROR1 antibody drug conjugate (ADC) zilovertamab vedotin and the BTKi nemtabrutinib have shown promising results as monotherapies in the treatment of hematological malignancies. In view of the high mortality of MCL, there is a high unmet medical need for new therapies.

### SUMMARY OF THE INVENTION

An aspect of the present invention relates to a method of treating a B-cell malignancy, comprising co-administering to a subject in need thereof an immunoconjugate of Formula (I),

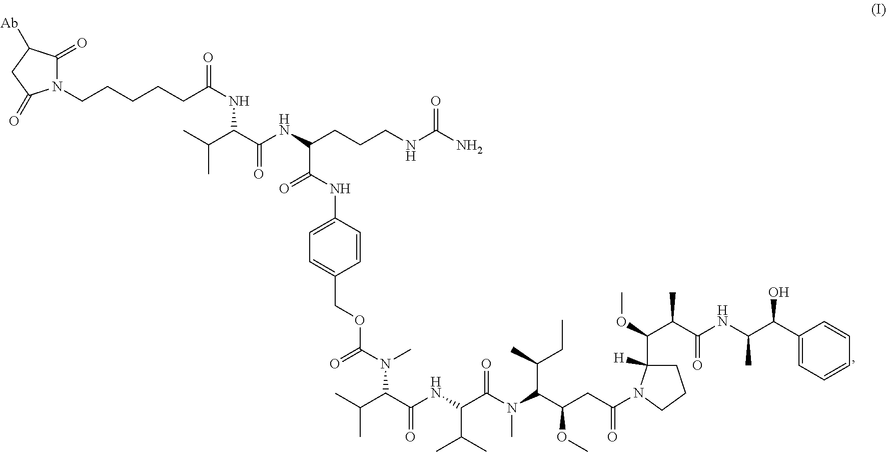

and a compound of Formula (II), or a pharmaceutically acceptable salt thereof,

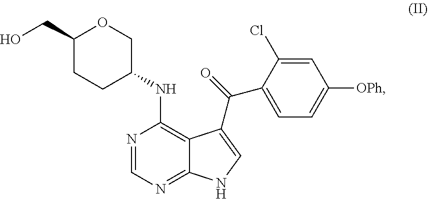 [SMILES: O=C(c1ccc(Oc2ccccc2)cc1Cl)c1c[nH]c2ncnc(N[C@@H]3CC[C@@H](CO)OC3)c12]

wherein the immunoconjugate comprises an antibody (Ab) conjugated to a drug moiety shown in Formula (I), and wherein the antibody comprises a heavy and light chain comprising the amino acid sequences of SEQ ID NOs: 1 and 2, respectively.

In some embodiments, the number of the drug moiety per antibody (DAR) is from 1 to 7. In some embodiments, the number of the DAR is from 3 to 6. In certain embodiments, the number of the DAR is from 3 to 5.

In some embodiments, the immunoconjugate of Formula (I) is administered to the subject at a dose from 0.25 to 4.00 mg/kg. In certain embodiments, the immunoconjugate of Formula (I) is administered to the subject at a dose of 2.00, 2.25, or 2.50 mg/kg.

In some embodiments, the compound of Formula (II) is administered to the subject at a dose of 5 to 75 mg per day. In certain embodiments, the compound of Formula (II) is administered to the subject at a dose of 65 mg per day.

In some embodiments, the immunoconjugate of Formula (I) is administered in three-week cycles and administered on Day 1 of each cycle at said dose. In some embodiments, the immunoconjugate of Formula (I) is administered in three-week cycles and administered on Days 1 and 8 of each cycle at said dose. In some embodiments, the immunoconjugate of Formula (I) is administered in four-week cycles and administered on Days 1, 8, and 15 of each cycle at said dose. In some embodiments, the number of cycles is 2 or more. In some embodiments, the number of cycles is 7 or more.

In some embodiments, the compound of Formula (II) is administered one, two, three, or four times per day. In some embodiments, the compound of Formula (II) is administered once per day.

In some embodiments, the compound of Formula (II) is administered every day for at least one day. In some embodiments, the compound of Formula (II) is administered every day for at least one week. In some embodiments, the compound of Formula (II) is administered every day for at least three weeks. In some embodiments, the compound of Formula (II) is administered every day for at least three months. In some embodiments, the compound of Formula (II) is administered every day for at least twelve months.

In some embodiments, the immunoconjugate of Formula (I) is administered intravenously. In some embodiments, the compound of Formula (II) is administered orally.

In some embodiments, the B-cell malignancy is an aggressive B-cell malignancy. In certain embodiments, the B-cell malignancy is a mantle cell lymphoma (MCL).

In some embodiments, the patient has been treated previously for the B-cell malignancy. In some embodiments, the B-cell malignancy is relapsed or refractory to treatment.

In some embodiments, co-administration of the immunoconjugate of Formula (I) and compound of Formula (II) results in one or more of the following: disease regression; delayed disease progression; prevention of disease recurrence or residual disease; decrease of the size of nodal or extranodal malignant masses; decrease of the malignant cell numbers in bone marrow and/or peripheral blood; decrease of malignant splenomegaly or hepatomegaly; improved anemia, neutropenia, or thrombocytopenia; ameliorated cutaneous manifestation; ameliorated disabling constitutional symptoms; and prolonged survival. In certain embodiments, co-administration of the immunoconjugate of Formula (I) and compound of Formula (II) results in complete disease eradication.

In some embodiments, the immunoconjugate of Formula (I) is zilovertamab vedotin. In some embodiments, the compound of Formula (II) is nemtabrutinib.

Another aspect of the present invention relates to an immunoconjugate of Formula (I) and a compound of Formula (II) for use in treating a B-cell malignancy in a subject according to any of the methods described herein.

In some embodiments, the immunoconjugate of Formula (I) is zilovertamab vedotin. In some embodiments, the compound of Formula (II) is nemtabrutinib.

Another aspect of the present invention relates to a use of an immunoconjugate of Formula (I) and a compound of Formula (II) for the manufacture of a medicament for treating a B-cell malignancy in a subject according to any of the methods described herein.

In some embodiments, the immunoconjugate of Formula (I) is zilovertamab vedotin. In some embodiments, the compound of Formula (II) is nemtabrutinib.

Another aspect of the present invention relates to a method of treating a B-cell malignancy, comprising co-administering to a subject in need thereof an immunoconjugate of Formula (I),

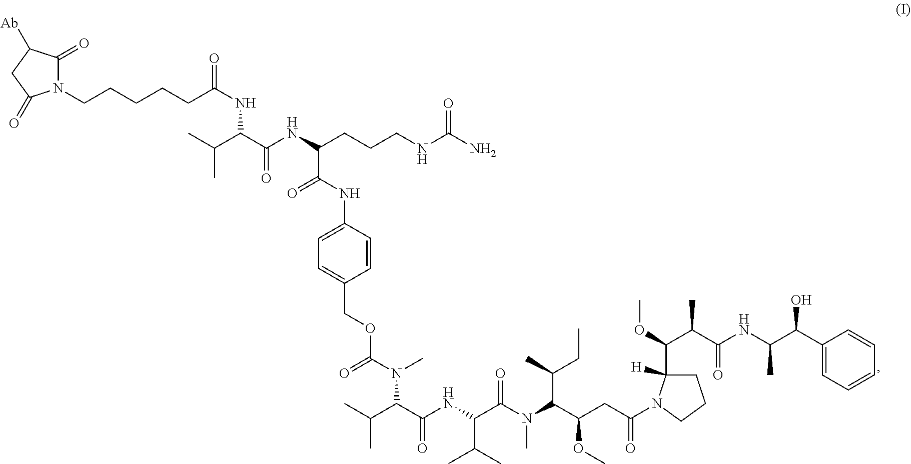

and a compound of Formula (II), or a pharmaceutically acceptable salt thereof,

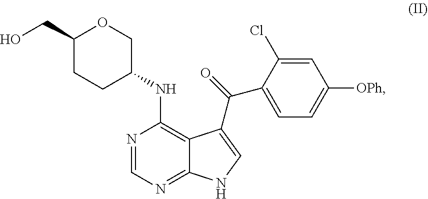 [SMILES: O=C(c1ccc(Oc2ccccc2)cc1Cl)c1c[nH]c2ncnc(N[C@@H]3CC[C@@H](CO)OC3)c12]

wherein the immunoconjugate comprises an antibody (Ab) conjugated to a drug moiety shown in Formula (I), wherein the number of the drug moiety per antibody (DAR) ranges from 3 to 5, wherein the antibody comprises a heavy and light chain comprising the amino acid sequences of SEQ ID NOs: 1 and 2, respectively, wherein the immunoconjugate of Formula (I) is administered to the subject in three-week cycles at a dose of 2.00, 2.25, or 2.50 mg/kg on Days 1 and 8 of each cycle, wherein the compound of Formula (II) is administered to the subject at a dose of 65 mg daily, and wherein the B-cell malignancy is a mantle cell lymphoma (MCL).

In some embodiments, the immunoconjugate of Formula (I) is zilovertamab vedotin. In some embodiments, the compound of Formula (II) is nemtabrutinib.

Another aspect of the present invention relates to a pharmaceutical composition comprising effective amounts of an immunoconjugate of Formula (I),

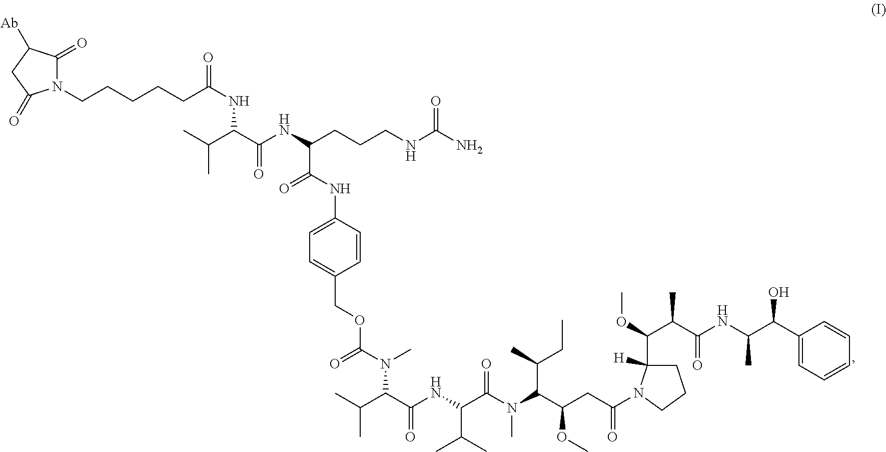

and a compound of Formula (II), or a pharmaceutically acceptable salt thereof,

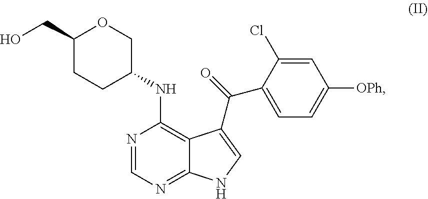 [SMILES: O=C(c1ccc(Oc2ccccc2)cc1Cl)c1c[nH]c2ncnc(N[C@@H]3CC[C@@H](CO)OC3)c12]

wherein the immunoconjugate comprises an antibody (Ab) conjugated to a drug moiety shown in Formula (I), wherein the number of the drug moiety per antibody (DAR) ranges from 3 to 5, wherein the antibody comprises a heavy and light chain comprising the amino acid sequences of SEQ ID NOs: 1 and 2, respectively; and a pharmaceutically acceptable carrier.

In some embodiments, the immunoconjugate of Formula (I) is zilovertamab vedotin. In some embodiments, the compound of Formula (II) is nemtabrutinib.

In some embodiments, the effective amount of zilovertamab vedotin is a dose of 2.00, 2.25, or 2.50 mg/kg. In some embodiments, the effective amount of nemtabrutinib is a dose of 65 mg.

Another aspect of the present invention relates to a kit comprising a pharmaceutical composition described herein and instructions for use in treating a B-cell malignancy.

### BRIEF DESCRIPTION OF THE DRAWINGS

FIG. 1 is a schematic depiction of the presently described treatment regimens.

### DETAILED DESCRIPTION OF THE INVENTION

The present invention provides treatment regimens using an ROR1 immunoconjugate and a BTK inhibitor. These co-administration regimens may be useful for the treatment of a variety of cancers that express ROR1 and are expected to be mediated or modulated by BTK signaling, e.g., B-cell malignancies. The combination of ROR1 immunoconjugates and BTK inhibitors has the potential to yield deeper and more prolonged responses in B-cell cancers such as MCL. In certain embodiments, the B-cell malignancy is a mantle cell lymphoma (MCL).

### 1. Immunoconjugates

An “antibody-drug conjugate,” or “ADC,” or “immunoconjugate,” refers to an antibody molecule or an antigen-binding fragment thereof that is covalently or non-covalently bonded, with or without a linker, to one or more biologically active molecule(s). The term “antibody” is used herein in the broadest sense and includes polyclonal and monoclonal antibodies, such as intact antibodies and functional (antigen-binding) fragments thereof. The term encompasses genetically engineered and/or otherwise modified forms of immunoglobulins, such as intrabodies, peptibodies, chimeric antibodies, fully human antibodies, humanized antibodies, and heteroconjugate antibodies, multi-specific (e.g., bispecific) antibodies, diabodies, triabodies, and tetrabodies, tandem di-scFv, and tandem tri-scFv. Unless otherwise indicated, the term encompasses intact or full-length antibodies, including antibodies of any class or subclass (e.g., IgG and sub-classes thereof such as IgG1, IgG2, IgG3, and IgG4; IgM; IgE; IgA; and IgD), as well as antibody fragments.

An antibody may include a heavy chain (or a polypeptide sequence derived therefrom) and a light chain (or a polypeptide sequence derived therefrom). The term “variable region” or “variable domain” refers to the domain of an antibody heavy or light chain that is involved in the antibody's binding to an antigen. The variable domains of the heavy chain and light chain (VH and VL, respectively) of a native antibody generally have similar structures, with each domain comprising four conserved framework regions and three complementarity-determining regions. A single VH or VL domain may sometimes be sufficient to confer all or a majority of the antigen-binding specificity of an antibody. Furthermore, antibodies that bind a particular antigen may be isolated by using a VH or VL domain from an antibody that binds the antigen to screen a library of complementary VL or VH domains, respectively. See, e.g., Portolano et al., J. Immunol. 150:880-887 (1993); Clarkson et al., Nature 352:624-628 (1991).

The terms “complementarity-determining region” and “CDR,” which are synonymous with “hypervariable region” or “HVR,” refer to subregions within the antibody variable domains, which confer the antibody's specificity and/or affinity for its antigen. In general, there are three CDRs in each heavy chain variable domain (HCDR1, HCDR2, and HCDR3) and three CDRs in each light chain variable domain (LCDR1, LCDR2, and LCDR3). “Framework regions” (“FRs”) refer to the non-CDR portions of the variable domains. In general, there are four FRs in each full-length heavy chain variable domain and four FRs in each full-length light chain variable domain. The precise amino acid sequence boundaries of a given CDR or FR can be readily determined using any of several well-known schemes, including those described by Kabat et al., 5th Ed., Public Health Service, National Institutes of Health, Bethesda, MD (1991) (“Kabat” numbering scheme); Al-Lazikani et al., JMB 273, 927-948 (1997) (“Chothia” numbering scheme); MacCallum et al., J. Mol. Biol. 262:732-745 (1996) (“contact” numbering scheme); Lefranc et al., Dev Comp Immunol. 27(1):55-77 (2003) (“IMGT” numbering scheme); and Honegger and Pluckthun, J Mol Biol, 309(3):657-70 (2001) (“Aho” numbering scheme). The boundaries of a given CDR or FR may vary depending on the scheme used for identification. For example, the Kabat scheme is based on sequence alignments, while the Chothia scheme is based on structural information. Numbering for both the Kabat and Chothia schemes is based upon the most common antibody region sequence lengths, with insertions accommodated by insertion letters, for example, “30a.” The two schemes place certain insertions and deletions (“indels”) at different positions, resulting in differential numbering. The contact scheme is based on analysis of complex crystal structures and is similar in many respects to the Chothia numbering scheme. Unless indicated otherwise, the CDRs of the antibodies referred to herein may be identified according to any of the Kabat, Chothia, IMGT, and contact methods.

An antigen-binding fragment of a full-length antibody may be used in making an immunoconjugate of the present invention. Examples of antibody fragments include, but are not limited to, Fv, Fab, Fab′, Fab′-SH, F(ab′)₂; recombinant IgG (rlgG) fragments; diabodies; linear antibodies; single-chain antibody molecules (e.g., scFv or sFv); single domain antibodies (e.g., sdAb, sdFv, nanobodies); and multi-specific antibodies formed from antibody fragments. In certain embodiments, the fragments are single-chain antibody fragments comprising a variable heavy chain region and/or a variable light chain region, such as scFvs.

The present immunoconjugates comprise antibodies or fragments thereof that are specific for human ROR1 and thus can serve as excellent targeting moieties for delivering the conjugated payloads to cells (e.g., ROR1-positive cells). In certain embodiments, an immunoconjugate used in a treatment regimen of the invention is an immunoconjugate described in WO 2018/237335. Shown below in Table 1 are SEQ ID NOs for the amino acid sequences of the heavy and light chain complementarity-determining regions (HCDRs and LCDRs), heavy and light chain variable domains (VH and VL), and heavy and light chains (HC and LC) of an exemplary anti-ROR1 antibody (i.e., Ab1) used in the immunoconjugates described herein.

TABLE 1
List of Ab1 Amino Acid Sequences

| SEQ | | |
|---|---|---|
| ID | | |
| NO. | Description | Amino Acid Sequence |
| 1 | Ab1 heavy | QVQLQESGPGLVKPSQTLSLTCTVSGYAFT |
| | chain | AYNIHWVRQAPGQGLEWMGSFDPYDGGSSY |
| | | NQKFKDRLTISKDTSKNQVVLTMTNMDPVD |
| | | TATYYCARGWYYFDYWGHGTLVTVSSASTK |
| | | GPSVFPLAPSSKSTSGGTAALGCLVKDYFP |
| | | EPVTVSWNSGALTSGVHTFPAVLQSSGLYS |
| | | LSSVVTVPSSSLGTQTYICNVNHKPSNTKV |
| | | DKKVEPKSCDKTHTCPPCPAPELLGGPSVF |
| | | LFPPKPKDTLMISRTPEVTCVVVDVSHEDP |
| | | EVKFNWYVDGVEVHNAKTKPREEQYNSTYR |
| | | VVSVLTVLHQDWLNGKEYKCKVSNKALPAP |
| | | IEKTISKAKGQPREPQVYTLPPSRDELTKN |
| | | QVSLTCLVKGFYPSDIAVEWESNGQPENNY |
| | | KTTPPVLDSDGSFFLYSKLTVDKSRWQQGN |
| | | VFSCSVMHEALHNHYTQKSLSLSPGK |
| 2 | Ab1 light | DIVMTQTPLSLPVTPGEPASISCRASKSIS |
| | chain | KYLAWYQQKPGQAPRLLIYSGSTLQSGIPP |
| | | RFSGSGYGTDFTLTINNIESEDAAYYFCQQ |
| | | HDESPYTFGEGTKVEIKRTVAAPSVFIFPP |
| | | SDEQLKSGTASVVCLLNNFYPREAKVQWKV |
| | | DNALQSGNSQESVTEQDSKDSTYSLSSTLT |
| | | LSKADYEKHKVYACEVTHQGLSSPVTKSFN |
| | | RGEC |
| 3 | Ab1 VH | QVQLQESGPGLVKPSQTLSLTCTVSGYAFT |
| | | AYNIHWVRQAPGQGLEWMGSFDPYDGGSSY |
| | | NQKFKDRLTISKDTSKNQVVLTMTNMDPVD |
| | | TATYYCARGWYYFDYWGHGTLVTVSS |
| 4 | Ab1 VL | DIVMTQTPLSLPVTPGEPASISCRASKSIS |
| | | KYLAWYQQKPGQAPRLLIYSGSTLQSGIPP |
| | | RFSGSGYGTDFTLTINNIESEDAAYYFCQQ |
| | | HDESPYTFGEGTKVEIK |
| 5 | Ab1 HCDR1 | GYAFTAYN |
| 6 | Ab1 HCDR2 | FDPYDGGS |
| 7 | Ab1 HCDR3 | GWYYFDY |
| 8 | Ab1 LCDR1 | KSISKY |
| 9 | Ab1 LCDR2 | SGS |
| 10 | Ab1 LCDR3 | QQHDESPY |

In some embodiments, the antibody or antibody fragment in the immunoconjugate specifically binds human ROR1, and its heavy and light chains respectively comprise:
a) the HCDRs 1-3 amino acid sequences in SEQ ID NO: 1, and the LCDRs 1-3 amino acid sequences in SEQ ID NO: 2;
b) HCDRs 1-3 comprising the amino acid sequences of SEQ ID NO: 5-7, respectively, and LCDRs 1-3 comprising the amino acid sequences of SEQ TD NOs: 8-10, respectively;
c) HCDRs 1-3 comprising residues 26-33, 51-58, and 97-105 of SEQ TD NO: 3, respectively, and LCDRs 1-3 comprising residues 27-32, 50-52, and 89-97 of SEQ TD NO: 4, respectively;
d) HCDRs 1-3 comprising residues 26-32, 52-57, and 99-105 of SEQ ID NO: 3, respectively, and LCDRs 1-3 comprising residues 24-34, 50-56, and 89-97 of SEQ ID NO: 4, respectively; or
e) HCDRs 1-3 comprising residues 31-35, 50-66, and 99-105 of SEQ ID NO: 3, respectively, and LCDRs 1-3 comprising residues 24-34, 50-56, and 89-97 of SEQ ID NO: 4, respectively.

In some embodiments, the amino acid sequences of the antibodies or fragments thereof of the immunoconjugates described herein may further comprise or include post-translational modifications thereof (e.g., C-terminal lysine clipping or alpha amidation in the heavy chain, methionine oxidation and/or asparagine deamidation in the heavy chain, conversion of glutamine or glutamic acid to pyroglutamate in the N-terminus of the variable domain, glycosylation in the antibody Fc region, etc.) which may occur when recombinantly expressed in host cells (e.g., CHO cells), or during purification and/or storage of the antibody, fragment, or immunoconjugate. In some embodiments, the C-terminal lysine of SEQ ID NO:1 is present. In other embodiments, the C-terminal lysine of SEQ ID NO:1 is absent. In some embodiments, the N-terminal glutamine of SEQ ID NO:3 is not converted to pyroglutamate. In other embodiments, the N-terminal glutamine of SEQ ID NO:3 is converted to pyroglutamate.

In some embodiments of an immunoconjugate described herein, the antibody can be conjugated to the cytotoxic agent via a linker. In some embodiments, the linker is a cleavable linker. A cleavable linker refers to a linker that comprises a cleavable moiety and is typically susceptible to cleavage under in vivo conditions. In certain embodiments, the linker may comprise a dipeptide, such as a valine-citrulline (Val-Cit or VC) linker. In certain embodiments, the linker is attached to a cysteine residue on the antibody.

In some embodiments, the conjugation of the linker/payload to the antibody or fragment may be formed through reaction with a maleimide group, which may also be referred to as a maleimide spacer. In certain embodiments, the maleimide group is maleimidocaproyl (mc); thus, the linker/payload is conjugated to the antibody or fragment through reaction between a residue on the antibody or fragment and the me group in the linker precursor.

In some embodiments, the linker may include a benzoic acid or benzyloxy group, or a derivative thereof. In some embodiments, the linker includes a para-amino-benzyloxycarbonyl (PAB) group.

In some embodiments, the linkage between the antibody and payload or drug components of the immunoconjugate may be formed through reaction of the components with a linker comprising a maleimide group, a peptide moiety, and/or a benzoic acid (e.g., PAB) group, in any combination. In certain embodiments, the maleimide group is maleimidocaproyl (mc). In certain embodiments, the peptide group is Val-Cit (VC). In certain embodiments, the linker comprises a Val-Cit-PAB group. In certain embodiments, the conjugation of the linker to the antibody or fragment may be formed from an mc-Val-Cit group. In certain embodiments, the linkage between the antibody or fragment and the drug moiety may be formed from an mc-Val-Cit-PAB group.

Linkers can be conjugated to the anti-ROR1 antibodies and antigen-binding fragments of the current disclosure in multiple ways. Generally, a linker and a cytotoxic moiety are synthesized and conjugated before attachment to an antibody. One method of attaching a linker-drug conjugate to an antibody involves reduction of solvent-exposed disulfides with dithiothreitol (DTT) or tris (2-carboxyethyl)phosphine (TCEP), followed by modification of the resulting thiols with maleimide-containing linker-drug moieties (e.g., 6-maleimidocaproyl-valine-citrulline-p-aminobenzyloxycarbonyl (mc-VC-PAB)). A native antibody contains 4 inter-chain disulfide bonds and 12 intra-chain disulfide bonds, as well as unpaired cysteines. Thus, antibodies modified in this way can comprise greater than one linker-drug moiety per antibody. In certain embodiments, the immunoconjugates each comprise at least 1, 2, 3, 4, 5, 6, 7, 8, 9, or 10 linker/drug moieties. In certain embodiments, the immunoconjugates each comprise one or more (e.g., 1 to 10, 1 to 9, 1 to 8, 1 to 7, 1 to 6, 1 to 5, 1 to 4, 1 to 3, or 1 to 2) linker/drug moieties. In cases where the linker is branched and can each attach to multiple drug moieties, the ratio of the drug moiety to the antibody will be higher than using an unbranched linker.

In some embodiments, a suitable cytotoxic agent for use in an immunoconjugate described herein may be, e.g., an anti-tubulin agent such as an auristatin. In certain embodiments, the cytotoxic agent is monomethyl auristatin E (MMAE).

In some embodiments, an immunoconjugate described herein is constructed as follows:
*MAL: maleimide chemistry (MAL).

| mAb | Conjugation | Linker Components | | Payload |
|---|---|---|---|---|
| anti-ROR1 antibody | MAL (mc) | VC | PAB | MMAE |

The anti-ROR1 antibody may be an anti-ROR1 antibody described herein, e.g., an antibody with a heavy chain amino acid sequence of SEQ ID NO: 1 and a light chain amino acid sequence of SEQ ID NO: 2.

In particular embodiments, an immunoconjugate used in the treatment regimens of the invention has the structure of Formula (I):

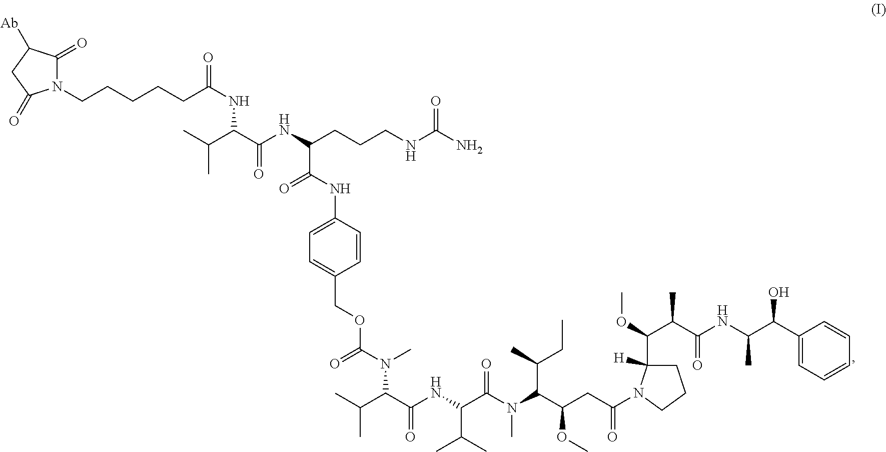

In some embodiments, the antibody (Ab) is Ab1, which has a heavy chain amino acid sequence of SEQ ID NO: 1 and a light chain amino acid sequence of SEQ ID NO: 2. In some embodiments, the immunoconjugate may have a DAR of 3-6 and is referred to herein as “ADC-A.” In some embodiments, the payload MMAE is conjugated to Ab1 through cysteine residue(s) in the antibody polypeptide chains. In some embodiments, the immunoconjugate is selected from the disclosure of WO 2018/237335. In some embodiments, the immunoconjugate is zilovertamab vedotin.

### 2. BTK Inhibitors

The term “BTK inhibitor” or “BTKi” refers to a compound or pharmaceutically acceptable salts thereof, capable of inhibiting BTK. In particular embodiments, the BTKi is a compound of Formula (II):

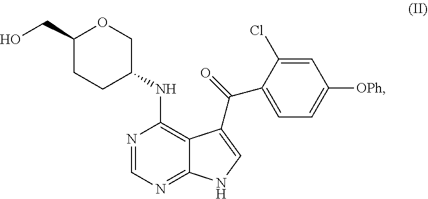 [SMILES: O=C(c1ccc(Oc2ccccc2)cc1Cl)c1c[nH]c2ncnc(N[C@@H]3CC[C@@H](CO)OC3)c12]

or pharmaceutically acceptable salts thereof, capable of inhibiting BTK. In some embodiments, the compound of Formula (II) is nemtabrutinib. In some embodiments, the compound of Formula (II) is useful for the treatment of diseases and disorders associated with modulation of BTK. In some embodiments, the BTK is wild-type BTK. In other embodiments, the BTK is a mutant BTK.

In some embodiments, the compound of Formula (II) inhibits the kinase activity of a mutant BTK, such as a drug-resistant mutant BTK harboring a drug-resistance mutation (e.g., C481S mutation). In certain embodiments, the BTK mutation is a C481S mutation.

The compound of Formula (II) may form salts which are also within the scope of this application. The present invention is directed in part to a compound of Formula (II) and pharmaceutically acceptable salts thereof.

The compound of the present application, and pharmaceutically acceptable salts, tautomers, prodrugs, and polymorphs of the compound of Formula (II) thereof, can exist in a solvated form with other solvent molecules or in an unsolvated form.

“Solvate” means solvent addition forms that contain either stoichiometric or non-stoichiometric amounts of solvent. Some compounds or salts have a tendency to trap a fixed molar ratio of solvent molecules in the crystalline solid state, thus forming a solvate. If the solvent is water the solvate formed is a hydrate; and if the solvent is alcohol, the solvate formed is an alcoholate. Hydrates are formed by the combination of one or more molecules of water with one molecule of the substance in which the water retains its molecular state as H₂O.

All stereoisomers (for example, geometric isomers, optical isomers and the like) of the present compound (including those of the salts, solvates, esters and prodrugs of the compound as well as the salts, solvates and esters of the prodrugs), such as those which may exist due to asymmetric carbons on various substituents, including enantiomeric forms (which may exist even in the absence of asymmetric carbons), rotameric forms, atropisomers, and diastereomeric forms, are contemplated within the scope of this application, as are positional isomers (such as, for example, 4-pyridyl and 3-pyridyl). For example, if a compound of Formula (II) incorporates a double bond or a fused ring, both the cis- and trans-forms, as well as mixtures, are embraced within the scope of the application. Individual stereoisomers of the compound of the application may, for example, be substantially free of other isomers, or may be admixed, for example, as racemates or with all other, or other selected, stereoisomers. The chiral centers of the present application can have the S or R configuration as defined by the IUPAC 1974 Recommendations. The use of the term “salt,” “solvate,” “ester,” “prodrug,” and the like, is intended to equally apply to the salt, solvate, ester and prodrug of enantiomers, stereoisomers, rotamers, tautomers, positional isomers, racemates or prodrugs of the compound.

The term “isomer” refers to compounds that have the same composition and molecular weight but differ in physical and/or chemical properties. The structural difference may be in constitution (geometric isomers) or in the ability to rotate the plane of polarized light (stereoisomers). With regard to stereoisomers, the compound of Formula (II) may have one or more asymmetric carbon atom and may occur as racemates, racemic mixtures or as individual enantiomers or diastereomers.

In the present specification, the structural formula of the compound represents a certain isomer for convenience in some cases, but the present application includes all isomers, such as geometrical isomers, optical isomers based on an asymmetrical carbon, stereoisomers, tautomers, and the like.

“Isomerism” means compounds that have identical molecular formulae but differ in the sequence of bonding of their atoms or in the arrangement of their atoms in space. Isomers that differ in the arrangement of their atoms in space are termed “stereoisomers.” Stereoisomers that are not mirror images of one another are termed “diastereoisomers,” and stereoisomers that are non-superimposable mirror images of each other are termed “enantiomers” or sometimes optical isomers. A mixture containing equal amounts of individual enantiomeric forms of opposite chirality is termed a “racemic mixture.”

The compound of Formula (II) may contain asymmetric or chiral centers, and, therefore, exist in different stereoisomeric forms. It is intended that all stereoisomeric forms of the compound of the application as well as mixtures thereof, including racemic mixtures, form part of the present application. In addition, the present application embraces all geometric and positional isomers. For example, if a compound of the application incorporates a double bond or a fused ring, both the cis- and trans-forms, as well as mixtures, are embraced within the scope of the application. Each compound herein disclosed includes all the enantiomers that conform to the general structure of the compound. The compound may be in a racemic or enantiomerically pure form, or any other form in terms of stereochemistry. The assay results may reflect the data collected for the racemic form, the enantiomerically pure form, or any other form in terms of stereochemistry.

A carbon atom bonded to four non-identical substituents is termed a “chiral center.” “Chiral isomer” means a compound with at least one chiral center. Compounds with more than one chiral center may exist either as an individual diastereomer or as a mixture of diastereomers, termed “diastereomeric mixture.” When one chiral center is present, a stereoisomer may be characterized by the absolute configuration (R or S) of that chiral center. Absolute configuration refers to the arrangement in space of the substituents attached to the chiral center. The substituents attached to the chiral center under consideration are ranked in accordance with the Sequence Rule of Cahn, Ingold and Prelog. (Cahn et al., Angew. Chem. Inter. Edit. 1966, 5, 385; errata 511; Cahn et al., Angew. Chem. 1966, 78, 413; Cahn and Ingold, J. Chem. Soc. 1951 (London), 612; Cahn et al., Experientia 1956, 12, 81; Cahn, J. Chem. Educ. 1964, 41, 116). “Geometric isomer” means the diastereomers that owe their existence to hindered rotation about double bonds. These configurations are differentiated in their names by the prefixes cis and trans, or Z and E, which indicate that the groups are on the same or opposite side of the double bond in the molecule according to the Cahn-Ingold-Prelog rules.

In another embodiment of the application, the compound of Formula (II) is an enantiomer. In some embodiments the compound is the (S)-enantiomer. In other embodiments the compound is the (R)-enantiomer. In yet other embodiments, the compound of Formula (II) may be (+) or (−) enantiomers. The compound may contain more than one stereocenter.

In another embodiment of the application, the compound of Formula (II) are diastereomers. In some embodiments, the compound are the syn-diastereomer. In other embodiments, the compound are the anti-diastereomer.

Diastereomeric mixtures can be separated into their individual diastereomers on the basis of their physical chemical differences by methods well known to those skilled in the art, such as, for example, by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereomers and converting (e.g., hydrolyzing) the individual diastereomers to the corresponding pure enantiomers. Enantiomers can also be separated by use of a chiral HPLC column.

It is also possible that the compound of the application may exist in different tautomeric forms, and all such forms are embraced within the scope of the application. Also, for example, all keto-enol and imine-enamine forms of the compound are included in the application.

“Tautomer” is one of two or more structural isomers that exist in equilibrium and is readily converted from one isomeric form to another. This conversion results in the formal migration of a hydrogen atom accompanied by a switch of adjacent conjugated double bonds. Tautomers exist as a mixture of a tautomeric set in solution. In solid form, usually one tautomer predominates. In solutions where tautomerization is possible, a chemical equilibrium of the tautomers will be reached. The exact ratio of the tautomers depends on several factors, including temperature, solvent and pH. The concept of tautomers that are interconvertable by tautomerizations is called tautomerism.

Of the various types of tautomerism that are possible, two are commonly observed. In keto-enol tautomerism a simultaneous shift of electrons and a hydrogen atom occurs. Ring-chain tautomerism arises as a result of the aldehyde group (—CHO) in a sugar chain molecule reacting with one of the hydroxy groups (—OH) in the same molecule to give it a cyclic (ring-shaped) form as exhibited by glucose.

Common tautomeric pairs are: ketone-enol, amide-nitrile, lactam-lactim, amide-imidic acid tautomerism in heterocyclic rings (e.g., in nucleobases such as guanine, thymine and cytosine), amine-enamine and enamine-imine. (Pyrrolopyrimidinyl)methanone-(Pyrrolopyrimidinyl)methanol tautomeric pairs are included in the present application:

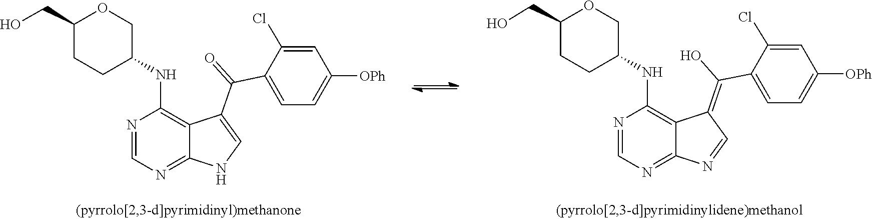 [SMILES: O=C(c1ccc(Oc2ccccc2)cc1Cl)c1c[nH]c2ncnc(N[C@@H]3CC[C@@H](CO)OC3)c12 ; OC[C@@H]1CC[C@@H](Nc2ncnc3c2/C(=C(\O)c2ccc(Oc4ccccc4)cc2Cl)C=N3)CO1]

The compound can be converted to N-oxides by treatment with an oxidizing agent (e.g., 3-chloroperoxybenzoic acid (m-CPBA) and/or hydrogen peroxides) to afford other compounds. Thus, all shown and claimed nitrogen-containing compounds are considered, when allowed by valency and structure, to include both the compound as shown and its N-oxide derivative (which can be designated as N→O or N+—O⁻). Furthermore, in other instances, the nitrogens in the compound can be converted to N-hydroxy or N-alkoxy compounds. For example, N-hydroxy compounds can be prepared by oxidation of the parent amine by an oxidizing agent such as m CPBA. All shown and claimed nitrogen-containing compounds are also considered, when allowed by valency and structure, to cover both the compounds as shown and its N-hydroxy (i.e., N—OH) and N-alkoxy (i.e., N—OR, wherein R is substituted or unsubstituted C1-C 6 alkyl, C1-C6 alkenyl, C1-C6 alkynyl, 3-14-membered carbocycle or 3-14-membered heterocycle) derivatives. The term “prodrug,” as used in this application, means a compound which is convertible in vivo by metabolic means (e.g., by hydrolysis) to the compound of Formula (II).

Since prodrugs are known to enhance numerous desirable qualities of pharmaceuticals (e.g., solubility, bioavailability, manufacturing, etc.) the compound of Formula (II), or pharmaceutically acceptable salts thereof, can be delivered in prodrug form. Thus, the present application is intended to cover prodrugs of the compound of Formula (II), or a pharmaceutically acceptable salt thereof, methods of delivering the same and compositions containing the same. “Prodrugs” are intended to include any covalently bonded carriers that release an active parent drug of the present application in vivo when such prodrug is administered to a mammalian subject. Prodrugs are prepared by modifying functional groups present in the compound in such a way that the modifications are cleaved, either in routine manipulation or in vivo, to the parent compound. Prodrugs include compounds of the application wherein a hydroxyl or amino, group is bonded to any group that, when the prodrug of the present application is administered to a mammalian subject, it cleaves to form a free hydroxyl or free amino group, respectively. Examples of prodrugs include, but are not limited to, acetate, formate, and benzoate derivatives of alcohol and amine functional groups in the compound of Formula (II) or a pharmaceutically acceptable salt thereof.

The term “crystal polymorphs,” “polymorphs,” or “crystal forms” means crystal structures in which a compound (or a salt or solvate thereof) can crystallize in different crystal packing arrangements, all of which have the same elemental composition. Different crystal forms usually have different X-ray diffraction patterns, infrared spectral, melting points, density hardness, crystal shape, optical and electrical properties, stability and solubility. Recrystallization solvent, rate of crystallization, storage temperature, and other factors may cause one crystal form to dominate. Crystal polymorphs of the compound of Formula (II) can be prepared by crystallization under different conditions.

The application also comprehends isotopically-labeled immunoconjugates and compounds, which are identical to those recited in the each of the formulae described herein, but for the fact that one or more atoms are replaced by an atom having an atomic mass or mass number different from the atomic mass or mass number most commonly found in nature. Examples of isotopes that can be incorporated into the immunoconjugate of Formula (I) or compound of Formula (II) include isotopes of hydrogen, carbon, nitrogen, fluorine, such as 3H, 11C, 14C, 2H, and 18F. The immunoconjugate of Formula (I) and the compound of Formula (II), or pharmaceutically acceptable salts thereof, that contain the aforementioned isotopes and/or other isotopes of other atoms are within the scope of the present application. Isotopically-labeled immunoconjugates and compounds of the present application, for example those into which radioactive isotopes such as 3H and 14C are incorporated, are useful in drug and/or substrate tissue distribution assays. Tritiated, i.e., 3H, and carbon-14, i.e., 14C, isotopes are useful for their ease of preparation and detectability. 11C and 18F isotopes are useful in positron emission tomography (PET). PET is useful in brain imaging. Further, substitution with heavier isotopes such as deuterium, i.e., 2H, can afford certain therapeutic advantages resulting from greater metabolic stability, for example increased in vivo half-life or reduced dosage requirements and, hence, may be preferred in some circumstances. Isotopically labeled immunoconjugates of Formula (I) and compounds of Formula (II), or pharmaceutically acceptable salts thereof, can generally be prepared by carrying out the procedures described herein, by substituting a readily available isotopically labeled reagent for a non-isotopically labeled reagent.

The compound of the present application can be prepared in a number of ways well known to those skilled in the art of organic synthesis, such as the method described in U.S. Pat. No. 9,630,968.

### 3. Treatment Regimens

The present invention provides treatment regimens comprising co-administering to a patient in need thereof an effective amount of an ROR1 immunoconjugate and a BTK inhibitor. In certain embodiments the ROR1 immunoconjugate is an immunoconjugate of Formula (I) and the BTK inhibitor is a compound of Formula (II), or a pharmaceutically acceptable salt thereof. These co-administration regimens may be useful for the treatment of a variety of cancers that express ROR1 and are expected to be mediated or modulated by BTK signaling. In certain embodiments, the cancer is a B-cell malignancy. In certain embodiments, the B-cell malignancy is a mantle cell lymphoma (MCL).

The term “administer,” “administered,” “administering,” or “administration” as used in this application refers to directly providing an effective amount of an ROR1 immunoconjugate and/or a BTK inhibitor to a patient or subject in need thereof. The term “co-administer,” “co-administered,” “co-administering,” or “co-administration” as used in this application refers to providing effective amounts of an ROR1 immunoconjugate and a BTK inhibitor to a patient or subject in need thereof. The co-administered ROR1 immunoconjugate and BTK inhibitor may be administered on the same day or on separate days of the same administration cycle. In some embodiments, the co-administered ROR1 immunoconjugate and BTK inhibitor are administered on the same day of an administration cycle. In some embodiments, the co-administered ROR1 immunoconjugate and BTK inhibitor are administered on separate days of an administration cycle.

A “patient” or “subject” is a mammal, e.g., a human, mouse, rat, guinea pig, dog, cat, horse, cow, pig, or non-human primate, such as a monkey, chimpanzee, baboon, or rhesus.

An “effective amount” or “therapeutically effective amount” when used in connection with an immunoconjugate, compound, or pharmaceutical composition is an amount effective for treating or preventing a disease in a subject as described herein.

The term “treating” with regard to a subject, refers to improving at least one symptom of the subject's disorder. Treating includes curing, improving, or at least partially ameliorating the disorder.

As used herein, “preventing” or “prevent” describes reducing or eliminating the onset of the symptoms or complications of the disease, condition or disorder.

The term “disorder” is used in this application to mean, and is used interchangeably with, the terms disease, condition, or illness, unless otherwise indicated.

As used herein, the term “BTK-mediated” or “BTK-modulated” diseases or disorders means any disease or other deleterious condition in which BTK, or a mutant thereof, is known to play a role. Accordingly, another embodiment of the present application relates to treating or lessening the severity of one or more diseases in which BTK, or a mutant thereof, is known to play a role.

Specifically, the present application relates to a method of treating or lessening the severity of a disease or condition selected from a proliferative disorder or an autoimmune disorder, wherein said method comprises co-administering to a patient in need thereof an immunoconjugate of Formula (I) and compound of Formula (II), or pharmaceutically acceptable salts thereof, or a composition according to the present application.

As used herein, the term “cell proliferative disorder” refers to conditions in which unregulated or abnormal growth, or both, of cells can lead to the development of an unwanted condition or disease, which may or may not be cancerous. Exemplary cell proliferative disorders of the application encompass a variety of conditions wherein cell division is deregulated. Exemplary cell proliferative disorder include, but are not limited to, neoplasms, benign tumors, malignant tumors, pre-cancerous conditions, in situ tumors, encapsulated tumors, metastatic tumors, liquid tumors, solid tumors, immunological tumors, hematological tumors, cancers, carcinomas, leukemias, lymphomas, sarcomas, and rapidly dividing cells. The term “rapidly dividing cell” as used herein is defined as any cell that divides at a rate that exceeds or is greater than what is expected or observed among neighboring or juxtaposed cells within the same tissue. A cell proliferative disorder includes a precancer or a precancerous condition. A cell proliferative disorder includes cancer. Preferably, the methods provided herein are used to treat or alleviate a symptom of cancer. The term “cancer” includes solid tumors, as well as hematologic tumors and/or malignancies. A “precancer cell” or “precancerous cell” is a cell manifesting a cell proliferative disorder that is a precancer or a precancerous condition. A “cancer cell” or “cancerous cell” is a cell manifesting a cell proliferative disorder that is a cancer. Any reproducible means of measurement may be used to identify cancer cells or precancerous cells. Cancer cells or precancerous cells can be identified by histological typing or grading of a tissue sample (e.g., a biopsy sample). Cancer cells or precancerous cells can be identified through the use of appropriate molecular markers.

As used herein, “relapsed” and “refractory” are defined as follows: Relapsed disease is disease progression following at least one line or prior therapy and Refractory disease is failure to achieve a complete response (CR) or partial response (PR) with at least one line of prior therapy. In some embodiments, the relapsed disease, such as a relapsed hematological malignancy, is disease progression following the most recent therapy. In some embodiments, refractory disease, such as a relapsed hematological malignancy, is failure to achieve CR or PR with the most recent therapy.

Exemplary non-cancerous conditions or disorders include, but are not limited to, rheumatoid arthritis; inflammation; autoimmune disease; chronic Graft Versus Host Disease (cGVHD), lymphoproliferative conditions; acromegaly; rheumatoid spondylitis; osteoarthritis; gout, other arthritic conditions; sepsis; septic shock; endotoxic shock; gram-negative sepsis; toxic shock syndrome; asthma; adult respiratory distress syndrome; chronic obstructive pulmonary disease; chronic pulmonary inflammation; inflammatory bowel disease; Crohn's disease; psoriasis; eczema; ulcerative colitis; pancreatic fibrosis; hepatic fibrosis; acute and chronic renal disease; irritable bowel syndrome; pyresis; restenosis; cerebral malaria; stroke and ischemic injury; neural trauma; Alzheimer's disease; Huntington's disease; Parkinson's disease; acute and chronic pain; allergic rhinitis; allergic conjunctivitis; chronic heart failure; acute coronary syndrome; cachexia; malaria; leprosy; leishmaniasis; Lyme disease; Reiter's syndrome; acute synovitis; muscle degeneration, bursitis; tendonitis; tenosynovitis; herniated, ruptures, or prolapsed intervertebral disk syndrome; osteopetrosis; thrombosis; restenosis; silicosis; pulmonary sarcosis; bone resorption diseases, such as osteoporosis; graft-versus-host reaction; Multiple Sclerosis; lupus; fibromyalgia; AIDS and other viral diseases such as Herpes Zoster, Herpes Simplex I or II, influenza virus and cytomegalovirus; and diabetes mellitus.

Exemplary cancers include, but are not limited to, adrenocortical carcinoma, AIDS-related cancers, AIDS-related lymphoma, anal cancer, anorectal cancer, cancer of the anal canal, appendix cancer, childhood cerebellar astrocytoma, childhood cerebral astrocytoma, basal cell carcinoma, skin cancer (non-melanoma), biliary cancer, extrahepatic bile duct cancer, intrahepatic bile duct cancer, bladder cancer, urinary bladder cancer, bone and joint cancer, osteosarcoma and malignant fibrous histiocytoma, brain cancer, brain tumor, brain stem glioma, cerebellar astrocytoma, cerebral astrocytoma/malignant glioma, ependymoma, medulloblastoma, supratentorial primitive neuroectodermal tumors, visual pathway and hypothalamic glioma, breast cancer, bronchial adenomas/carcinoids, carcinoid tumor, gastrointestinal, nervous system cancer, nervous system lymphoma, central nervous system cancer, central nervous system lymphoma, cervical cancer, childhood cancers, chronic lymphocytic leukemia, chronic myelogenous leukemia, chronic myeloproliferative disorders, colon cancer, colorectal cancer, cutaneous T-cell lymphoma, lymphoid neoplasm, mycosis fungoides, Seziary Syndrome, endometrial cancer, esophageal cancer, extracranial germ cell tumor, extragonadal germ cell tumor, extrahepatic bile duct cancer, eye cancer, intraocular melanoma, retinoblastoma, gallbladder cancer, gastric (stomach) cancer, gastrointestinal carcinoid tumor, gastrointestinal stromal tumor (GIST), germ cell tumor, ovarian germ cell tumor, gestational trophoblastic tumor glioma, head and neck cancer, hepatocellular (liver) cancer, Hodgkin lymphoma, hypopharyngeal cancer, intraocular melanoma, ocular cancer, islet cell tumors (endocrine pancreas), Kaposi Sarcoma, kidney cancer, renal cancer, kidney cancer, laryngeal cancer, acute lymphoblastic leukemia, acute myeloid leukemia, chronic lymphocytic leukemia, chronic myelogenous leukemia, hairy cell leukemia, lip and oral cavity cancer, liver cancer, lung cancer, non-small cell lung cancer, small cell lung cancer, AIDS-related lymphoma, B-cell non-Hodgkin lymphoma, non-Hodgkin lymphoma, primary central nervous system lymphoma, Waldenstrom's macroglobulinemia, medulloblastoma, melanoma, intraocular (eye) melanoma, merkel cell carcinoma, mesothelioma malignant, mesothelioma, metastatic squamous neck cancer, mouth cancer, cancer of the tongue, multiple endocrine neoplasia syndrome, mycosis fungoides, myelodysplastic syndromes, myelodysplastic/myeloproliferative diseases, chronic myelogenous leukemia, acute myeloid leukemia, multiple myeloma, chronic myeloproliferative disorders, nasopharyngeal cancer, neuroblastoma, oral cancer, oral cavity cancer, oropharyngeal cancer, ovarian cancer, ovarian epithelial cancer, ovarian low malignant potential tumor, pancreatic cancer, islet cell pancreatic cancer, paranasal sinus and nasal cavity cancer, parathyroid cancer, penile cancer, pharyngeal cancer, pheochromocytoma, pineoblastoma and supratentorial primitive neuroectodermal tumors, pituitary tumor, plasma cell neoplasm/multiple myeloma, pleuropulmonary blastoma, prostate cancer, rectal cancer, renal pelvis and ureter, transitional cell cancer, retinoblastoma, rhabdomyosarcoma, salivary gland cancer, ewing family of sarcoma tumors, Kaposi Sarcoma, soft tissue sarcoma, uterine cancer, uterine sarcoma, skin cancer (non-melanoma), skin cancer (melanoma), merkel cell skin carcinoma, small intestine cancer, soft tissue sarcoma, squamous cell carcinoma, stomach (gastric) cancer, supratentorial primitive neuroectodermal tumors, testicular cancer, throat cancer, thymoma, thymoma and thymic carcinoma, thyroid cancer, transitional cell cancer of the renal pelvis and ureter and other urinary organs, gestational trophoblastic tumor, urethral cancer, endometrial uterine cancer, uterine sarcoma, uterine corpus cancer, vaginal cancer, vulvar cancer, chronic lymphocytic leukemia (CLL), Richter's Transformation, small lymphocytic lymphoma (SLL), diffuse large B-cell lymphoma (DLBCL), follicular lymphoma (FL), mantle cell lymphoma (MCL), primary central nervous system (CNS) lymphoma, secondary central nervous system (CNS) lymphoma, marginal zone lymphoma (MZL), Waldenstrom's macroglobulinemia (WM), acute myeloid leukemia (AML), multiple myeloma (MM), pediatric sarcoma, pediatric brain tumors, and Wilm's Tumor. In some embodiments, the cancer is a B-cell malignancy. In certain embodiments, the B-cell malignancy is a mantle cell lymphoma (MCL).

In some embodiments, a ROR1 immunoconjugate of Formula (I) is administered at a dose of 0.25 to 10 mg/kg, e.g., 0.25 to 4 mg/kg. For example, the immunoconjugate of Formula (I) may be administered at a dose of 0.25 0.5, 0.75, 1, 1.25, 1.5, 1.75, 2, 2.25, 2.5, 2.75, 3, 3.25, 3.5, 3.75, 4, 4.25, 4.5, 4.75, 5, 5.5, 6, 6.5, 7, 8, 9, or 10 mg/kg, or any combination thereof for multiple doses. In certain embodiments, the immunoconjugate of Formula (I) is administered at a dose of 2.00, 2.25, or 2.50 mg/kg.

In some embodiments, the immunoconjugate of Formula (I) is administered in repeated cycles of 1, 2, 3, 4, 5, 6, 7, 8, 9, 10, 11, 12, 13, 14, 15, or 16 weeks. In certain embodiments, the immunoconjugate of Formula (I) is administered in three-week cycles. In certain embodiments, the immunoconjugate of Formula (I) is administered in four-week cycles. The treatment regimen may comprise 1, 2, 3, 4, 5, 6, 7, 8, 9, 10, 11, 12, 13, 14, 15, 16, 17, 18, 19, 20, or more cycles of administration (e.g., 3 or more cycles, or 4 or more cycles). In certain embodiments, the immunoconjugate of Formula (I) is administered on one, two, three, four, five, six, or seven days of the cycle. The days of administration may be consecutive or may have one, two, three, four, five, or six days, one week, two weeks, three weeks, or four weeks, or any combination thereof, between them. In particular embodiments, the immunoconjugate of Formula (I) is administered on Day 1 only of each cycle (e.g., a three-week cycle). In particular embodiments, the immunoconjugate of Formula (I) is administered on Days 1 and 8 of each cycle (e.g., a three-week cycle). In particular embodiments, the immunoconjugate of Formula (I) is administered on Days 1, 8, and 15 of each cycle (e.g., a four-week cycle).

The immunoconjugate of Formula (I) may be administered initially according to a dosage regimen described herein and subsequently according to a different dosage regimen described herein (e.g., to increase or decrease the frequency of administration). In some embodiments, the immunoconjugate of Formula (I) is administered weekly during the first 1, 2, 3, 4, 5, 6, 7, 8, 9, 10, 11, or 12 weeks, then every 3 weeks thereafter. In certain embodiments, the immunoconjugate of Formula (I) is administered weekly during the first 2, 3, 4, 5, or 6 weeks, and then every 3 weeks. In certain embodiments, the immunoconjugate of Formula (I) is administered weekly during the first 1, 2, 3, 4, 5, or 6 weeks, and then every 4 weeks.

In some embodiments, a dosage regimen described herein achieves an immunoconjugate plasma Cₘₐₓ of at least 10, 15, 20, 25, 30, 35, 40, 45, 50, 55, or 60 μg/mL in the patient. In some embodiments, a dosage regimen described herein achieves an immunoconjugate plasma area under the concentration-time curve (AUC) of at least 500, 750, 1000, 1250, 1500, 1750, 2000, 2250, 2500, 2750, 3000, 3250, or 3500 hour·μg/mL in the patient.

In some embodiments, a dosage regimen described herein maintains immunoconjugate occupancy of the ROR1 receptor of at least 30%, 40%, 50%, 60%, 70%, 75%, 80%, 85%, 90%, or 95% in the patient. In some embodiments, a dosage regimen described herein maintains at least 50% immunoconjugate occupancy of the ROR1 receptor for at least 20, 30, 40, 50, 60, 70, 80, or 90% of the time. In some embodiments, a dosage regimen described herein maintains at least 75% immunoconjugate occupancy of the ROR1 receptor for at least 20, 30, 40, 50, 60, 70, 80, or 90% of the time. In some embodiments, a dosage regimen described herein maintains at least 90% immunoconjugate occupancy of the ROR1 receptor for at least 0.5, 1, 1.5, 2, 2.5, 3, 3.5, 4, 4.5, or 5% of the time.

The immunoconjugate of Formula (I) may be administered via parenteral administration. As used herein, “parenteral administration” of an immunoconjugate includes any route of administration characterized by physical breaching of a tissue of a subject and administration of the immunoconjugate through the breach in the tissue, thus generally resulting in the direct administration into the blood stream, into muscle, or into an internal organ. Parenteral administration thus includes, but is not limited to, administration of an immunoconjugate by injection of the immunoconjugate, by application of the immunoconjugate through a surgical incision, by application of the immunoconjugate through a tissue-penetrating non-surgical wound, and the like. In particular, parenteral administration is contemplated to include, but is not limited to, subcutaneous, intraperitoneal, intramuscular, intrasternal, intravenous, intraarterial, intrathecal, intraventricular, intraurethral, intracranial, intratumoral, and intrasynovial injection or infusions; and kidney dialytic infusion techniques. Regional perfusion is also contemplated. In some embodiments, the infusion may be administered by one route (e.g., intravenously) for initial doses and then be administered by another route for subsequent doses.

In certain embodiments, the immunoconjugate of Formula (I) is administered by intravenous (IV) infusion. The IV infusion may take place over a period of about 0.1 to about 4 hours (e.g., about 5, 10, 15, 20, 25, 30, 35, 40, 45, 50, 55, 60, 65, 70, 75, 80, 85, 90, 120, or 180). In particular embodiments, the infusion time is 30 minutes. Infusion times may be extended as necessary to accommodate individual patient tolerance of treatment. Where the immunoconjugate is administered in more than one dose, in some embodiments, the infusion time for the first dose is longer than the infusion time for subsequent doses, or alternatively, the infusion time for the first dose is shorter than the infusion time for subsequent doses.

In some embodiments, immunoconjugate of Formula (I) is administered for a period until the patient shows no symptoms of the diseases or disorders. In some embodiments, immunoconjugate of Formula (I) is administered for a period until the patient is cured of the diseases or disorders. In some embodiments, immunoconjugate of Formula (I) is administered for a period until the patient shows resistance to the immunoconjugate. In some embodiments, immunoconjugate of Formula (I) is administered for a period until the patient shows side effects that would require discontinuation of the therapy with the immunoconjugate.

In some embodiments, the immunoconjugate of Formula (I) is co-administered to a patient or subject with another therapeutic agent. In some embodiments, the immunoconjugate of Formula (I) is co-administered with a BTK inhibitor. In certain embodiments, the BTK inhibitor is a compound of Formula (II), or a pharmaceutically acceptable salt thereof.

In some embodiments, the compound of Formula (II), or a pharmaceutically acceptable salt thereof, is administered in an amount from about 5 mg to about 75 mg per day.

In some embodiments, the compound of Formula (II), or a pharmaceutically acceptable salt thereof, is administered in an amount of at least 5 mg per day. In some embodiments, the compound of Formula (II), or a pharmaceutically acceptable salt thereof, is administered in an amount of at least 10 mg per day. In some embodiments, the compound of Formula (II), or a pharmaceutically acceptable salt thereof, is administered in an amount of at least 15 mg per day. In some embodiments, the compound of Formula (II), or a pharmaceutically acceptable salt thereof, is administered in an amount of at least 20 mg per day. In some embodiments, the compound of Formula (II), or a pharmaceutically acceptable salt thereof, is administered in an amount of at least 25 mg per day. In some embodiments, the compound of Formula (II), or a pharmaceutically acceptable salt thereof, is administered in an amount of at least 30 mg per day.

In some embodiments, the compound of Formula (II), or a pharmaceutically acceptable salt thereof, is administered in an amount of at least 35 mg per day. In some embodiments, the compound of Formula (II), or a pharmaceutically acceptable salt thereof, is administered in an amount of at least 40 mg per day. In some embodiments, the compound of Formula (II), or a pharmaceutically acceptable salt thereof, is administered in an amount of at least 45 mg per day. In some embodiments, the compound of Formula (II), or a pharmaceutically acceptable salt thereof, is administered in an amount of at least 50 mg per day. In some embodiments, the compound of Formula (II), or a pharmaceutically acceptable salt thereof, is administered in an amount of at least 55 mg per day. In some embodiments, the compound of Formula (II), or a pharmaceutically acceptable salt thereof, is administered in an amount of at least 60 mg per day. In some embodiments, the compound of Formula (II), or a pharmaceutically acceptable salt thereof, is administered in an amount of at least 65 mg per day. In some embodiments, the compound of Formula (II), or a pharmaceutically acceptable salt thereof, is administered in an amount of at least 70 mg per day. In some embodiments, the compound of Formula (II), or a pharmaceutically acceptable salt thereof, is administered in an amount of at least 75 mg per day. For any of the doses disclosed herein, in some embodiments, the compound of Formula (II), or a pharmaceutically acceptable salt thereof, is administered one, two, three, or four times per day. For example, the compound is administered once per day. For example, the compound of Formula (II), or a pharmaceutically acceptable salt thereof, is administered twice per day. For example, the compound of Formula (II), or a pharmaceutically acceptable salt thereof, is administered three times per day. For example, the compound of Formula (II), or a pharmaceutically acceptable salt thereof, is administered four times per day. For example, the compound of Formula (II), or a pharmaceutically acceptable salt thereof, is administered once per day in an amount from about 5 mg to about 75 mg per day. For example the compound of Formula (II), or a pharmaceutically acceptable salt thereof, is administered twice per day in an amount from about 5 mg to about 75 mg per day. For example the compound of Formula (II), or a pharmaceutically acceptable salt thereof, is administered three times per day in an amount from about 5 mg to about 75 mg per day. For example, the compound of Formula (II), or a pharmaceutically acceptable salt thereof, is administered four times per day in an amount from about 5 mg to about 75 mg per day. In certain embodiments, the compound of Formula (II), or a pharmaceutically acceptable salt thereof, is administered once per day in an amount of about 65 mg per day.

In some embodiments, the compound of Formula (II), or a pharmaceutically acceptable salt thereof, is administered every day (i.e., daily) for at least one, two, three, four, five, six, seven, eight, nine, or ten days. In some embodiments, the compound of Formula (II), or a pharmaceutically acceptable salt thereof, is administered every day for at least one, two, three, four, five, six, seven, eight, nine, or ten weeks. In some embodiments, the compound of Formula (II), or a pharmaceutically acceptable salt thereof, is administered for at least one day per week, at least two days per week, at least three days per week, at least four days per week, at least five days per week, or at least six days per week.

In some embodiments, the compound of Formula (II), or a pharmaceutically acceptable salt thereof, is administered for one day per week, two days per week, three days per week, four days per week, five days per week, six days per week, or seven days per week. In some embodiments, the compound of Formula (II), or a pharmaceutically acceptable salt thereof, is administered on consecutive days. In some embodiments, the compound of Formula (II), or a pharmaceutically acceptable salt thereof, is administered on alternate days.

In some embodiments, the compound of Formula (II), or a pharmaceutically acceptable salt thereof, is administered for a period of at least one week, two weeks, three weeks, four weeks, five weeks, six weeks, seven weeks, eight weeks, nine weeks, or ten weeks.

In some embodiments, the compound of Formula (II), or a pharmaceutically acceptable salt thereof, is administered for a period of at least three months, four months, five months, six months, seven months, eight months, nine months, ten months, eleven months, twelve months, eighteen months, twenty-four months, three years, four years, or five years.

A total dose for a treatment interval is generally at least 0.05 g/kg body weight, more generally at least 0.2 g/kg, 0.5 g/kg, 1 g/kg, 10 g/kg, 100 g/kg, 0.25 mg/kg, 1.0 mg/kg, 2.0 mg/kg, 5.0 mg/ml, 10 mg/kg, 25 mg/kg, 50 mg/kg or more.

The compound of Formula (II), or a pharmaceutically acceptable salt thereof, may be administered by doses, e.g., daily, 1-7 times per week, weekly, bi-weekly, tri-weekly, every four weeks, every five weeks, every 6 weeks, monthly, bimonthly, quarterly, semiannually, annually, etc. Doses may be administered, e.g., intravenously, subcutaneously, topically, orally, nasally, rectally, intramuscular, intracerebrally, intraspinally, or by inhalation. In certain embodiments, the doses are administered intravenously. In certain embodiments, the doses are administered subcutaneously. In certain embodiments, the doses are administered orally.

In some embodiments, the compound of Formula (II), or a pharmaceutically acceptable salt thereof, is administered for a period until the patient shows no symptoms of the diseases or disorders. In some embodiments, the compound of Formula (II), or a pharmaceutically acceptable salt thereof, is administered for a period until the patient is cured of the diseases or disorders. In some embodiments, the compound of Formula (II), or a pharmaceutically acceptable salt thereof, is administered for a period until the patient shows resistance to the compound. In some embodiments, the compound of Formula (II), or a pharmaceutically acceptable salt thereof, is administered for a period until the patient shows side effects that would require discontinuation of the therapy with the compound.

In some embodiments, the subject is fasting for at least 1 hour, 2 hours, 3 hours, 4 hours, 6 hours, 8 hours, 12 hours before the compound is administered. In some embodiments, the subject needs to fast for at least 1 hour, 2 hours, 3 hours, 4 hours, 6 hours, 8 hours, 12 hours after the compound is administered.

It is understood that the treatment regimens of the invention may be methods of treatment as described herein, the immunoconjugate of Formula (I) and the compound of Formula (II), or a pharmaceutically acceptable salt thereof, as described herein for use in a treatment regimen described herein, or use of the immunoconjugate of Formula (I) and the compound of Formula (II), or a pharmaceutically acceptable salt thereof, as described herein for the manufacture of a medicament for use in a treatment regimen described herein.

The application also includes pharmaceutical compositions comprising an effective amount of the immunoconjugate of Formula (I) and the compound of Formula (II), or a pharmaceutically acceptable salt thereof, as disclosed herein, and a pharmaceutically acceptable carrier. The term “carrier,” as used in this application, encompasses carriers, excipients, and diluents and means a material, composition or vehicle, such as a liquid or solid filler, diluent, excipient, solvent or encapsulating material, involved in carrying or transporting a pharmaceutical agent from one organ, or portion of the body, to another organ, or portion of the body of a subject.

### 4. Patient Selection

The treatment regimens of the invention may be used to treat a patient with cancer. In some embodiments, a treatment regimen of the invention includes the step of selecting a patient with a cancer described herein. In certain embodiments, the patient may have been treated previously for said cancer, and/or has a cancer that is relapsed or is refractory to one or more (or all) existing treatments for said cancer.

In some embodiments, the treatment regimen is administered to a human patient, e.g., an adult patient (≥18 years of age), an adolescent patient (≥12 to 17 years of age), or a pediatric patient (<18 years of age). In some embodiments, the treatment regimen is administered to an adult patient (≥18 years of age) with adequate performance status and organ function who (i) has a histologically confirmed advanced hematological cancer; and/or (ii) has a malignancy that is unlikely to be responsive to established therapies known to provide clinical benefit, or has developed an intolerance to established therapies known to provide clinical benefit. In certain embodiments, the patient meets both criteria.

“Treat,” “treating,” and “treatment” refer to a method of alleviating or abrogating a biological disorder and/or at least one of its attendant symptoms. As used herein, to “alleviate” a disease, disorder or condition means reducing the severity and/or occurrence frequency of the symptoms of the disease, disorder, or condition. Further, references herein to “treatment” include references to curative, palliative and prophylactic treatment. Treatment of cancer encompasses inhibiting cancer growth (including causing partial or complete cancer regression), inhibiting cancer progression or metastasis, preventing cancer recurrence or residual disease, and/or prolonging the patient's survival.

In some embodiments, the patient to be treated with a treatment regimen of the invention has a ROR1-expressing cancer. The ROR1-expressing cancer can be determined by any suitable method of determining gene or protein expression, for example, by histology, flow cytometry, radiopharmaceutical methods, RT-PCR, or RNA-Seq. The cancer cells used for the determination may be obtained through tumor biopsy or through collection of circulating tumor cells. In certain embodiments, if an antibody-based assay such as flow cytometry or immunohistochemistry is used, ROR1-expressing cancers are any cancers with cells that show anti-ROR1 antibody reactivity greater than that of an isotype control antibody. In certain embodiments, if an RNA-based assay is used, ROR1-expressing cancers are those that show an elevated level of ROR1 RNA compared to a negative control cell or cancer that does not express ROR1.

In certain embodiments, the patient has a hematological malignancy, such as a lymphoid malignancy. In certain embodiments, the patient has a solid tumor. The patient may have a cancer selected from, e.g., lymphoma, non-Hodgkin lymphoma, chronic lymphocytic leukemia (CLL), small lymphocytic lymphoma (SLL), marginal zone lymphoma (MZL), marginal cell B-cell lymphoma, Burkitt lymphoma (BL), mantle cell lymphoma (MCL), follicular lymphoma (FL), diffuse large B-cell lymphoma (DLBCL), a non-Hodgkin lymphoma that has undergone Richter's transformation, T cell leukemia, T cell lymphoma (e.g., T cell non-Hodgkin lymphoma), lymphoplasmacytic lymphoma (LPL), Waldenstrom macroglobulinemia (WM), acute myeloid leukemia (AML), acute lymphocytic leukemia (ALL), hairy cell leukemia (HCL), myeloma, multiple myeloma (MM), sarcoma (e.g., osteosarcoma, Ewing sarcoma, rhabdomyosarcoma, soft-tissue sarcoma, or uterine sarcoma), brain cancer, glioblastoma, astrocytoma, medulloblastoma, craniopharyngioma, ependymoma, neuroblastoma, head and neck cancer, nasopharyngeal cancer, thyroid cancer, breast cancer (e.g., ER/PR-positive breast cancer, HER2-positive breast cancer, or triple-negative breast cancer), lung cancer (e.g., non-small cell lung cancer or small cell lung cancer), malignant mesothelioma, bile duct/gall bladder cancer (e.g., cholangiocarcinoma), colon cancer, colorectal cancer, esophageal cancer, stomach cancer, gastric cancer, gastrointestinal stromal tumors (GIST), liver (hepatocellular) cancer, pancreatic cancer, renal cell carcinoma, bladder cancer, prostate cancer, cervical cancer, endometrial cancer, ovarian cancer, testicular cancer, epithelial squamous cell cancer, melanoma, adrenocortical carcinoma, gastrointestinal carcinoid tumors, islet cell tumors, pancreatic neuroendocrine tumors, neuroendocrine carcinoma of the skin (Merkel cell carcinoma), and pheochromocytoma. In certain embodiments, the patient has a cancer that is refractory to other therapeutics (e.g., triple negative breast cancer).

In particular embodiments, the patient has MCL. In certain embodiments, the patient has PET-positive disease, defined as 4-5 on the Lugano 5-point scale, verified locally by an investigator at the time of Screening. In certain embodiments, the patient has radiographically measurable disease per the Lugano Response Criteria, as assessed locally by the investigator, with at least 1 nodal lesion (nonirradiated) that is >1.5 cm in the long axis, regardless of length of the short axis, or extranodal lesion of ≥1.0 cm in the long and short axis.

In certain embodiments, the patient may have been treated previously for said cancer, and/or has a cancer that is relapsed or is refractory to one or more (e.g., all) existing treatments for said cancer. In some embodiments, the patient is resistant to or has relapsed on treatment with ibrutinib, acalabrutinib, autologous hematopoietic stem cell transplantation, bendamustine, bortezomib, brentuximab vedotin, carmustine, chimeric antigen receptor T (CAR-T) cells, cisplatin, copanlisib, cyclophosphamide, cytarabine, daratumumab, dexamethasone, doxorubicin, etoposide, gemcitabine, idelalisib, lenalidomide, melphalan, methotrexate, methylprednisolone, mosunetuzumab, obinutuzumab, ofatumumab, oxaliplatin, pinatuzumab, polatuzumab, rituximab, prednisone, radiotherapy, venetoclax, vincristine, or any combination thereof (e.g., any combination of prior treatment agents found in the Examples). In certain embodiments, the patient was not previously treated with a non-covalent BTK inhibitor.

### 5. Treatment Outcomes

In some embodiments, a treatment regimen of the invention results in one or more of the following: disease regression; delayed disease progression; prevention of disease recurrence or residual disease; decrease of the size of nodal or extranodal malignant masses; decrease of the malignant cell numbers in bone marrow and/or peripheral blood; decrease of malignant splenomegaly or hepatomegaly; improved anemia, neutropenia, or thrombocytopenia; ameliorated cutaneous manifestation; ameliorated disabling constitutional symptoms (e.g., fever, headache, unexplained fatigue, malaise, lethargy, weight loss or gain, weakness, arthralgia (joint pain), myalgia (muscle pain), shaking, chills, nausea, vomiting, loss of appetite, abdominal pain, changes in bowel function, backaches, difficulty sleeping, and difficulty breathing); and prolonged survival.

In some embodiments, a treatment regimen of the invention results in complete disease eradication.

In some embodiments, a treatment regimen of the invention results in an improvement in a primary efficacy endpoint. In some embodiments, a primary efficacy endpoint is the achievement of objective response (OR), meaning a best overall response of a partial response (PR) or complete response (CR), defined according to the Lugano Response Criteria as assessed by the investigator. This assessment includes calculating the size of lymph nodes, extranodal lesions, spleen, and liver by computed tomography (CT) and/or magnetic resonance imaging (MRI), whole body metabolic imaging with fluorodeoxyglucose-positron emission tomography (FDG-PET), and clinical findings, e.g., physical examination and biopsy results, when these are available and appropriate.

A patient is determined to have achieved CR when: target nodes/nodal masses regress to ≤1.5 cm in the longest diameter (LDi) and no extranodal sites of disease remain (i.e., a PET-based response score of 1, 2, or 3); there is no evidence of new lesions or non-target lesions; previously enlarged organs regress to normal size; and bone marrow appears normal by PET and CT/MRI. A patient is determined to have achieved PR when: there is a ≥50% decrease from baseline in the sum of products of diameters (SPD) of target lymph nodes and extranodal sites (i.e., a PET-based response score of 4 or 5 without new lesions and a reduced overall uptake score compared to baseline measurements); there is no evidence of disease progression; the spleen regresses to ≥50% in excess length; there is no evidence of new lesions; and residual uptake is higher than normal in the bone marrow but lower than baseline, as determined by PET.

In some embodiments, a treatment regimen of the invention does not result in abnormalities in one or more (e.g., any one, two, three, four, five, or six) of the following: urine, serum, blood, systolic blood pressure, diastolic blood pressure, pulse, body temperature, blood oxygen saturation, and electrocardiography (ECG) readings. In certain embodiments, a treatment regimen of the invention does not result in abnormalities of any of the above.

In some embodiments, a treatment regimen of the invention does not result in detectable levels of circulating immunoconjugate-reactive antibodies in the patient's serum.

In certain embodiments, a treatment regimen of the invention does not result in dose-limiting toxicity (DLT). In some embodiments, a treatment regimen of the invention does not result in an adverse event (AE) or a serious adverse event (SAE).

An AE is any untoward medical occurrence in a clinical study participant, temporally associated with the use of study intervention, whether or not considered related to the study intervention.

An AE can therefore be any unfavorable and unintended sign (including an abnormal laboratory finding), symptom, or disease (new or exacerbated) temporally associated with the use of a study intervention.

An SAE is defined as any untoward medical occurrence that, at any dose: results in death; is life-threatening; requires inpatient hospitalization or prolongation of existing hospitalization; results in persistent or significant disability/incapacity; or results in a congenital anomaly/birth defect in offspring of a participant taking the product.

It is understood that the treatment regimens described herein may be methods of treatment as described herein, an immunoconjugate and/or a compound as described herein for use in a treatment regimen described herein, or use of an immunoconjugate and/or a compound as described herein for the manufacture of a medicament for a treatment regimen described herein.

### 6. Articles of Manufacture and Kits

The present invention also provides articles of manufacture, e.g., kits, comprising one or more containers (e.g., single-use or multi-use containers) containing a pharmaceutical composition of an immunoconjugate of Formula (I) described herein at a dose described herein, a pharmaceutical composition of a compound of Formula (II) described herein at a dose described herein, optionally an additional biologically active molecule (e.g., another therapeutic agent), and instructions for use according to a treatment regimen described herein. The immunoconjugate, compound, and additional biologically active molecule can be packaged together or separately in suitable packing such as a vial or ampule made from non-reactive glass or plastic.

In some embodiments, the vial or ampule holds a liquid containing the immunoconjugate or a lyophilized powder comprising the immunoconjugate; the liquid or lyophilized powder may optionally include the additional therapeutic agent or biologically active molecule. In certain embodiments, the vial or ampule holds a concentrated stock (e.g., 2×, 5×, 10× or more) of the immunoconjugate and optionally the biologically active molecule. In particular embodiments, a pharmaceutical composition of an immunoconjugate described herein (i.e., zilovertamab vedotin) is packaged in a single-use glass vial containing 50 mg, 100 mg, 150 mg, 200 mg, 250 mg, or 300 mg of the immunoconjugate (e.g., appropriate for use at a dose described herein, such as 2.00, 2.25, or 2.50 mg/kg).

In certain embodiments, the compound described herein (i.e., nemtabrutinib) is packaged in solid form (e.g., as a pill) at a 65 mg dosage. In certain embodiments, the solid form may optionally include the additional therapeutic agent or biologically active molecule.

In certain embodiments, the articles of manufacture such as kits include a medical device for administering the immunoconjugate, and/or biologically active molecule (e.g., a syringe and a needle), and/or an appropriate diluent (e.g., sterile water and normal saline). The present invention also includes methods for manufacturing said articles.

Unless the context requires otherwise, throughout the specification and claims, the word “comprise” and variations thereof, such as “comprises” and “comprising,” are to be construed in an open, inclusive sense, that is, as “including, but not limited to.” As used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise. As used herein the term “about” refers to a numerical range that is 10%, 5%, or 1% plus or minus from a stated numerical value within the context of the particular usage. Further, headings provided herein are for convenience only and do not interpret the scope or meaning of the claimed embodiments.

The details of the application are further set forth in the accompanying examples and claims described below. Although methods and materials similar or equivalent to those described herein can be used in the practice or testing of the present application, illustrative methods and materials are now described. Other features, objects, and advantages of the application will be apparent from the description and from the claims. In the specification and the appended claims, the singular forms also include the plural unless the context clearly dictates otherwise. Unless defined otherwise, all technical and scientific terms used herein have the same meaning as commonly understood by one of ordinary skill in the art to which this application belongs. All patents and publications cited in this specification are incorporated herein by reference in their entireties.

### EXAMPLES

The following examples illustrate representative embodiments of the present invention and are not meant to be limiting in any way.

### Example 1: Dose Finding Using a Modified Toxicity Probability Interval (mTPI) Design

Dose finding will follow the mTPI design with a target dose-limiting toxicity rate of 27%. The mTPI design is described in Ji Y, Li Y, Bekele B N. Dose-finding in phase 1 clinical trials based on toxicity probability intervals. Clin Trials 2007; 4:235-44. See also Ji Y, Wang S-J. Modified toxicity probability interval design: a safer and more reliable method than the 3+3 design for practical phase I trials. J Clin Oncol 2013; 31:1-12. Dose escalation and de-escalation decisions based on the mTPI design depend on the number of participants enrolled and number of DLTs observed at the current dose level. The mTPI design only applies to the initial DLT evaluation period.

A minimum of 3 participants are required at each dose. In Table 2 below, the columns indicate the numbers of participants treated at the current dose level, and the rows indicate the numbers of participants experiencing DLT. The entries of the table are the dose-finding decisions: E, S, and DU represent escalating the dose, staying at the same dose, and de-escalating the dose and excluding the dose from the study due to unacceptable toxicity, respectively. A dose is deemed as unacceptably toxic if the posterior probability of DLT rate being greater than 27% is greater than 80%. For example, if 0 of 3 participants at a given dose level develop a DLT, then the dose can escalate to the next level. If 2 or 3 of 3 participants develop a DLT, this indicates an unacceptable toxicity at this dose. The dose should be de-escalated, and the current dose will not be explored further. If 1 of 3 participants at a given dose level develop a DLT, then additional participants should be enrolled at that dose level following the rules described below.

When adding participants to a dose level in response to a “stay” decision, the number of additional participants to be enrolled is capped to minimize the exposure to a dose that may be unacceptably toxic (denoted as DU in Table 2). Second, to determine how many more participants can be enrolled at the dose level, one can count steps in diagonal direction (down and to the right) from the current cell to the first cell marked DU. For example, if 1 of 3 participants have experienced a DLT at a given dose level, no more than an additional 1 participant should be enrolled at this dose level until additional DLT data are available. This is because this dose level would be considered unacceptably toxic if the additional participant experiences a DLT (i.e., 2/4 participants with DLT in Table 2). The same principles will be applied whether 3, 4, 5, or 6 participants are initially enrolled at that dose level.

A DU decision at the lowest dose level will stop the study. An E decision at the highest acceptable dose level will result in staying at that level. During dose finding, it may be acceptable to de-escalate to an alternate dose for either compound that was not predefined and not previously studied if evaluation of toxicity at such a dose is desired. If this approach is taken, 3 to 6 new participants may be enrolled at the new alternate dose, and the aforementioned rules should be used to determine further enrollment at this dose level.

After 15 participants have been enrolled at any of the tested doses, dose finding will stop if the mTPI table indicates “S” for staying at current dose. Otherwise, up to 15 new participants may be enrolled at a lower dose if “DU” is indicated, or at a higher dose if “E” is indicated. To refine the selected dose, once an “S” is indicated in the mTPI, additional participants will be enrolled at the next lower dose level until a total of 15 participants have been enrolled at that dose level.

TABLE 2
Dose-Finding Rules Per mTPI Design

| Number of participants with | Number of Participants Evaluable for DLT at Current Dose | | | | | | | | | | | | |
|---|---|---|---|---|---|---|---|---|---|---|---|---|---|
| at least 1 DLT | 3 | 4 | 5 | 6 | 7 | 8 | 9 | 10 | 11 | 12 | 13 | 14 | 15 |
| 0 | E | E | E | E | E | E | E | E | E | E | E | E | E |
| 1 | S | S | S | S | E | E | E | E | E | E | E | E | E |
| 2 | DU | DU | S | S | S | S | S | S | S | S | E | E | E |
| 3 | DU | DU | DU | DU | DU | S | S | S | S | S | S | S | S |
| 4 | | DU | DU | DU | DU | DU | DU | DU | S | S | S | S | S |
| 5 | | | DU | DU | DU | DU | DU | DU | DU | DU | DU | DU | S |
| 6 | | | | DU | DU | DU | DU | DU | DU | DU | DU | DU | DU |
| 7 | | | | | DU | DU | DU | DU | DU | DU | DU | DU | DU |
| 8 | | | | | | DU | DU | DU | DU | DU | DU | DU | DU |
| 9 | | | | | | | DU | DU | DU | DU | DU | DU | DU |
| 10 | | | | | | | | DU | DU | DU | DU | DU | DU |
| 11 | | | | | | | | | DU | DU | DU | DU | DU |
| 12 | | | | | | | | | | DU | DU | DU | DU |
| 13 | | | | | | | | | | | DU | DU | DU |
| 14 | | | | | | | | | | | | DU | DU |
| 15 | | | | | | | | | | | | | DU |

DLT = dose-limiting toxicity; DU = de-escalate the dose and exclude the dose due to unacceptable toxicity; E = escalate to the next higher dose; mTPI = modified toxicity probability interval; S = stay at the current dose.
Note:
Target toxicity rate = 27% (inclusive).
Flat noninformative prior Beta (1,1) is used as a prior and ε1 = ε2 = 0.03.
Posterior probability of DLT rate for stopping = 80%.

### Example 2: Dose Escalation Study to Evaluate the Safety, Tolerability, and Efficacy of Zilovertamab Vedotin Co-Administered with Nemtabrutinib

The following describes a protocol for evaluating the safety, tolerability, and efficacy of zilovertamab vedotin and nemtabrutinib when co-administered to subjects with previously treated relapsed or refractory MCL. The primary objectives of the study are to evaluate the objective response rate (ORR) per Lugano Response Criteria. In addition, adverse events (AEs), AEs resulting in treatment discontinuation, and dose-limiting toxicities (DLTs) will be evaluated.

### 2.1 Patient Screening and Selection

### 2.1.1 Inclusion Criteria

Patients must meet the following selection criteria to be included in this study:
Male or female adult patients who are 18 years or older;
Biopsy proven and histologically confirmed MCL according to the 2016 WHO classification of neoplasms of the hematopoietic and lymphoid tissues;
Disease progressed during or relapsed after prior systemic therapy;
No prior exposure to a non-covalent BTKi;
PET-positive disease, defined as 4-5 on the Lugano 5-point scale, verified locally by the investigator at the time of Screening; and
Radiographically measurable disease per the Lugano Response Criteria, as assessed locally by the investigator, with at least 1 nodal lesion (nonirradiated) that is >1.5 cm in the long axis, regardless of length of the short axis, or extranodal lesion of ≥1.0 cm in the long and short axis.

### 2.1.2 Exclusion Criteria

Patients must be excluded from this study if the patient meets any of the following criteria:

### Medical Conditions

Has received solid organ transplant at any time;
Has clinically significant (i.e., active) cardiovascular disease, including cerebral vascular accident/stroke (<6 months prior to enrollment), myocardial infarction (<6 months prior to enrollment), unstable angina (<6 months prior to enrollment), congestive heart failure (New York Heart Association Classification Class ≥II), or serious cardiac arrhythmia requiring medication, or if patient presents with significant ECG abnormalities, including second degree atrial ventricular (AV) block type II, third degree AV block, bradycardia (ventricular rate less than 50 beats/min), or prolongation of QTc interval (per Fridericia's formula) >450 ms;
Has prior allogeneic stem cell transplant (allo-SCT), acute graft versus host disease (GVHD), or ongoing evidence of chronic GVHD manifesting as Grade ≥2 serum bilirubin, Grade ≥3 skin involvement, or Grade ≥3 diarrhea or requiring systemic immunosuppression for treatment/prophylaxis for their GVHD;
Has pericardial effusion or clinically significant pleural effusion;
Has ongoing Grade >1 peripheral neuropathy;
Has a demyelinating form of Charcot-Marie-Tooth disease;
Has a history of a second malignancy, unless potentially curative treatment has been completed with no evidence of malignancy for 2 years; this time requirement does not apply to participants who underwent successful definitive resection of basal cell carcinoma of the skin, squamous-cell carcinoma of the skin, superficial bladder cancer, in situ cervical cancer, or other in situ cancers;
Has follicular lymphoma (FL) which has transformed to a more aggressive type of lymphoma; if transformation is suspected, it must be ruled out prior to Screening; or
Has any clinically significant gastrointestinal abnormalities that might alter absorption.

### Prior/Concomitant Therapy

Has received prior therapy with a ROR1-directed therapy;
Has received prior therapy with a non-covalent BTKi;
Has contraindication to any of the study intervention components;
Has received prior systemic anticancer therapy, including investigational agents, within 5 half-lives or 4 weeks (if prior therapy was a monoclonal antibodies) or 2 weeks (small molecules like kinase inhibitors) prior to the first dose of study intervention; participants must have recovered from all AEs due to previous therapies to ≤Grade 1 or baseline; if the participant had a major operation, the participant must have recovered adequately from the procedure and/or any complications from the operation before starting study;
Has received prior radiotherapy within 28 days of start of study intervention; participants must have recovered from all radiation-related toxicities; a 1-week washout is permitted for palliative radiation (≤2 weeks of radiotherapy) to non-CNS disease;
Has received ongoing corticosteroid therapy exceeding 30 mg daily of prednisone equivalent; if taken, prednisone equivalent dosing of ≤30 mg daily must have been stable for at least 4 weeks prior to Day 1 of Cycle 1 (C1D1) unless corticosteroid treatment is required for lymphoma symptom control prior to C1D1; in that case, up to 100 mg per day of prednisone equivalent can be given for up to 5 days, and tumor assessments must have been completed prior to the start of corticosteroid treatment;
Has received a live or live-attenuated vaccine within 30 days before the first dose of study intervention; administration of killed vaccines are allowed;
Has received a strong inhibitor or inducer of CYP3A4 (including itraconazole, ketoconazole, posaconazole, or voriconazole) within 7 days prior to C1D1 or expected requirement for chronic use of a strong CYP3A4 inhibitor or inducer during the study intervention period and for 30 days after the last dose of study intervention; for subjects requiring antifungal prophylaxis/therapy, oral fluconazole or isavuconazonium can be considered; echinocandins (e.g., caspofungin, anidulafungin, or micafungin) are also acceptable, realizing the disadvantage of the requirement for intravenous administration; or
Is currently being treated with CYP2C8 substrates with a narrow therapeutic index (such as paclitaxel) or P-gp substrates with a narrow therapeutic index (such as digoxin); a washout period of at least 5 times the half-life after the last dose of any CYP2C8 or P-gp substrate is required for a participant to be eligible for study enrollment.

### Prior/Concurrent Clinical Study Experience

Is currently participating in or has participated in a study of an investigational agent or has used an investigational device within 4 weeks before the first dose of study intervention; participants who have entered the follow-up phase of an investigational study may participate as long as it has been 4 weeks after the last dose of the previous investigational agent.

### Diagnostic Assessments

Has known active central nervous system (CNS) lymphoma involvement or active CNS involvement by lymphoma; participants with prior CNS involvement are eligible if their CNS disease is in radiographic, cytological (for cerebrospinal fluid disease), and clinical remission;
Has an active infection requiring systemic therapy;
Has a known history of human immunodeficiency virus (HIV) infection;
Has an active hepatitis B virus (HBV) or hepatitis C virus (HCV) infection;
Has a history or current evidence of any condition, therapy, or laboratory abnormality, or other circumstance that might confound the results of the study, interfere with the participant's participation for the full duration of the study, such that it is not in the best interest of the participant to participate, in the opinion of the treating investigator; or
Has a known psychiatric or substance abuse disorder that would interfere with the participant's ability to cooperate with the requirements of the study.

### 2.2 Dose Levels and Dosing Schedule

After Screening, participants will receive zilovertamab vedotin co-administered with nemtabrutinib at one of the following three dose levels.

TABLE 3
Co-Administration Dose Levels

| Dose Level | Zilovertamab Vedotin | Nemtabrutinib |
|---|---|---|
| DL-1 | 2.0 mg/kg | 65 mg |
| DL0 | 2.25 mg/kg | 65 mg |
| DL1 | 2.5 mg/kg | 65 mg |

Zilovertamab vedotin will be administered intravenously in repeated 3-week cycles with a drug infusion on Days 1 and 8 of each cycle (Q2/3W) over a planned infusion time of ~30 minutes. Infusion times may be extended as necessary to accommodate individual subject tolerance of treatment. Nemtabrutinib will be co-administered on a daily basis (QD) over the entire study duration.

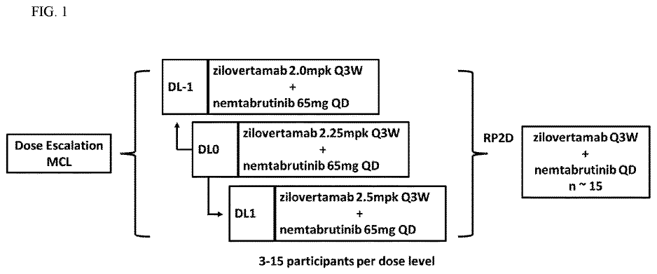

The starting dose level is DL0. If the number of DLTs observed in DL0 indicates an “E” based on the mTPI table, the dose will be escalated to DL1. If DL0 is not tolerated (“DU” on the mTPI table), the dose will be de-escalated to DL-1. A minimum of 3 participants are required for each dose level, with the potential to treat up to a maximum of 15 participants depending on dose decisions. A total of 15 participants will be also enrolled at the one dose level below which a stay decision is made based on the mTPI table. Enrollment will then be paused, and the totality of the data, including preliminary activity, safety, tolerability, and pharmacokinetic (PK), will be used to confirm the recommended Phase 2 dose (RP2D) of the combination. Once determined, an additional 15 participants will be enrolled to receive the RP2D. A schematic of the described protocol is shown in FIG. 1.

### 2.3 Efficacy Assessments

The primary efficacy endpoint for this study will be the achievement of objective response, meaning a best overall response of a partial response or complete response, defined according to the Lugano Response Criteria as assessed by the investigator. The Lugano Response Criteria, published in 2014 by Cheson, B. D., et al., is the revised response criteria in NHL and recommended by the current NCCN guidelines for B-cell lymphoma. See also National Comprehensive Cancer Network. NCCN clinical practice guidelines in oncology: B-cell lymphomas; version 4.2021. Plymouth Meeting (PA): National Comprehensive Cancer Network (NCCN); 2021. 306 p. This assessment includes anatomic imaging with CT or MRI (size assessments of lymph nodes, extranodal lesions, spleen, and liver), metabolic imaging (whole body assessment with FDG-PET), and clinical findings (physical examination and biopsy results), when these are available and appropriate.

Anatomic imaging may include CT, MRI or some combination of the two, with details specified in the Site Imaging Manual. CT is the most common modality used, and for the purposes of this document the term “CT” will be used to represent all anatomic imaging, no matter which imaging modality is used.

Before treatment (“baseline”), on CT all focal lesions (nodal and extranodal) will be classified as “target” (selected for quantitative assessment) and “non-target” (selected for qualitative assessment). The spleen will be assessed quantitatively (by measuring the vertical length), and the liver will be assessed qualitatively. The FDG-PET will be assessed using the 5-point scale (5PS), a method similar to the older Deauville criteria. See also Barrington S F, Mikhaeel N G, Kostakoglu L, Meignan M, Hutchings M, Mueller S P, et al. Role of imaging in the staging and response assessment of lymphoma: consensus of the International Conference on Malignant Lymphomas imaging working group. J Clin Oncol. 2014 Sep. 20; 32(27):3048-58. Erratum in: J Clin Oncol. 2016 Jul. 20; 34(21):2562. If biopsy is performed, or if there are any physical examination findings that cannot be evaluated by imaging, these should be documented.

After therapy has begun, response assessment will include anatomic response based on CT (when a CT is available), which includes target, non-target, and new focal lesions, as well as spleen and liver size assessment. Metabolic response, when an FDG-PET is available, will be based on the 5PS along with qualitative assessment of changes in FDG uptake from preceding timepoints. Anatomic response, metabolic response, and clinical information will be combined to produce the overall response for each timepoint. Evaluation response criteria are summarized in Table 4 below.

TABLE 4
Evaluation Response Evaluation Criteria
Complete Response (CR)

| CR | PET-Based Response - CMR | CT/MRI-Based Response - CR |
|---|---|---|
| Target lesions | Score 1, 2, or 3 | Target nodes/nodal masses regress |
| | | to ≤1.5 cm in LDi; no extranodal sites of |
| | | disease remain |
| Non-target lesions | Not applicable | Absent |
| Organ enlargement | Not applicable | Regress to normal |
| New lesions | None | None |
| Bone marrow | No evidence of FDG-avid disease in | Normal by morphology; if |
| | marrow | indeterminate, negative by |
| | | immunohistochemistry |

Partial Response (PR)

| PR | PET-Based Response - PMR | CT/MRI-Based Response - PR |
|---|---|---|
| Target lesions | Score 4 or 5 without new lesions | ≥50% decrease from baseline in SPD of |
| | Reduced overall uptake (extent | target lymph nodes and extranodal sites |
| | and/or intensity) compared to | (up to 6) |
| | baseline | |
| Non-target lesions | Not applicable | Anything other than progression |
| Organ enlargement | Not applicable | Spleen must have regressed by ≥50% in |
| | | excess length |
| New lesions | None | None |
| Bone marrow | Residual uptake higher than uptake | Not applicable |
| | in normal marrow but reduced | |
| | compared to baseline. | |
| | If there are persistent focal changes | |
| | in the marrow in the context of a | |
| | nodal response and without recent | |
| | growth factor use, perform biopsy, or | |
| | consider MRI, or an interval scan. | |

Stable Disease (SD)

| SD | PET- Based Response - SMD | CT/MRI-Based Response - SD |
|---|---|---|
| Target lesions | Score 4 or 5 (without new lesions) | <50% decrease from baseline in SPD |
| | No significant change in FDG uptake | of target lesions. |
| | from baseline or nadir | No lesion shows progression |
| Non-target lesions | Not applicable | No increase consistent with progression |
| Organ enlargement | Not applicable | No increase consistent with progression |
| New lesions | None | None |
| Bone marrow | No change from baseline | Not applicable |

Progressive Disease (PD)

| PD | PET-Based Response - PMD | CT/MRI-Based Response - PD |
|---|---|---|
| Target lesions | Score 4 or 5 with an increase in | Growth of any target lesion: |
| | overall uptake (extent and/or | Increase ≥50% from nadir PPD |
| | intensity) compared to nadir | and |
| | | Increase in LDi or SDi from |
| | | nadir of: ≥0.5 cm for lesions ≤2 |
| | | cm ≥1.0 cm for lesions >2 cm |
| | | and |
| | | Current LDi >1.5 cm for a lymph node |
| | | or ≥1.0 cm for an extranodal lesion |
| Non-target lesions | Not applicable | Clear progression of preexisting non- |
| | | target lesions |
| New lesions | New FDG-avid foci consistent | Regrowth of previously resolved |
| | with lymphoma rather than | lesions A new node >1.5 cm in LDi |
| | another cause, e.g., infection, | A new extranodal site of any size, as |
| | inflammation. | long as its presence is unequivocal and |
| | If uncertain regarding cause of new | attributable to lymphoma |
| | lesions, biopsy or interval scan may | |
| | be considered. | |
| Organ enlargement | Not applicable | When splenomegaly was already |
| | | present, the excess length must |
| | | increase by >50% (≥1 cm absolute |
| | | increase) from nadir. |
| | | If no prior splenomegaly, or prior |
| | | splenomegaly had resolved, spleen |
| | | length must increase by ≥2 cm |
| | | to >13 cm. |
| Bone marrow | New or recurrent FDG-avid foci, | New or recurrent involvement |
| | confirmed by biopsy | |

CMR = complete metabolic response; CR = complete response; CT = computed tomography; FDG = 2-fluorodeoxyglucose; LDi = longest diameter; MRI = magnetic resonance imaging; PD = progressive disease; PET = positron emission tomography; PMD = progressive metabolic disease; PMR = partial metabolic response; PPD = product of perpendicular diameters; PR = partial response; SD = stable disease; SDi = short axis diameter; SMD = stable metabolic disease; SPD = sum of products of diameters.

### 2.3.1 Anatomic Disease Assessment

Anatomic assessment specifically refers to the size of focal lesions or organs, assessed using computed tomography or magnetic resonance imaging. As stated previously, for simplicity the term “CT” will be used to represent all anatomic imaging.

### 2.3.2 Screening (Baseline) Assessment

### Documentation of Focal Lesions

All focal lesions caused by lymphoma are identified at baseline and classified as measurable or non-measurable. Up to 6 of the measurable lesions are selected to serve as “target” lesions, which are then followed quantitatively throughout the trial. All other focal lesions are documented as “non-target” lesions, and evaluated qualitatively thereafter.

### Measurable and Non-Measurable Lesions

Malignant lymph nodes (nodal lesions) are considered measurable if they are consistent with lymphoma, clearly and reproducibly measurable in 2 dimensions on an axial slice, and measure >1.5 cm in LDi when assessed by CT/MRI scan, irrespective of scanner type and slice thickness/interval. Extranodal lesions are considered measurable if they are consistent with lymphoma, clearly and reproducibly measurable in 2 dimensions on an axial slice, and are ≥1.0 cm in both LDi and short axis diameter (SDi) when assessed by CT/MRI scan, regardless of slice thickness. In lymphomas that are FDG-avid, a lesion must be PET positive (show FDG uptake greater than the surrounding tissue) to be measurable.

### Lesions Considered Non-Measurable Include:

Lymph nodes and nodal masses that are PET positive and considered consistent with lymphoma, but that do not meet the size and reproducibility requirements to be considered measurable, and lesions visible on PET but not CT;
PET-negative lesions which meet the size criteria for measurability, and are considered consistent with lymphoma, in lymphoma that shows FDG-avidity in other lesions;
Uni-dimensionally measurable lesions (clearly measurable in only one dimension);
Extranodal lesions which do not meet the requirements for measurability, but are considered to be clearly due to lymphoma;
Truly non-measurable/assessable sites of disease, including:
   Effusions and ascites;
   Bone lesions;
   Brain lesions, central nervous system (CNS) lesions, leptomeningeal disease;
   Mucosal lesions in the gastrointestinal tract; and
   Pleural, peritoneal or bowel wall thickening.

### Target and Non-Target Lesions

Up to up to 6 target lesions will be selected from among the measurable lesions and documented as target nodal and target extranodal lesions. Target lesions should be selected based on their size (largest lesions preferred) and suitability for reproducible measurements. Measurements of the longest diameter and short axis diameter should be made in the axial plane on the slice of the tumor with the longest in-plane diameter. Calculate the product of perpendicular diameters (PPD) for each target lesion and the sum of products of diameters for all target lesions. Non-target lesions will be all focal (nodal and extranodal) lesions that are consistent with lymphoma, but not chosen as target lesions, whether they were measurable or not. Once lesions are designated as target or non-target, those designations may not change during later assessments.

### Spleen Assessment at Baseline

Splenic involvement will be assessed quantitatively, as a separate category from the assessment of measurable or non-measurable focal lesions. The spleen length will be measured from cranial to caudal. All spleen measurements referred to hereafter will refer to this craniocaudal measurement. The spleen is considered normal if it is less than 13 cm, or if the spleen has been removed surgically. It is considered enlarged if it is greater than 13 cm in length. The portion of the measurement that exceeds 13 cm will be considered the abnormal portion.

### Liver Assessment at Baseline

Hepatic involvement will be assessed qualitatively, separately from the assessment of measurable or non-measurable disease. At baseline, the liver will be assessed qualitatively as either normal or enlarged, and should only be documented as enlarged if there is clear evidence (based on biopsy or imaging) that the enlargement is due to infiltration by lymphoma, and not to benign causes.

### 2.3.3 Post-Baseline Assessment

### Target Lesions

At every timepoint after screening, each target lesion is measured. Calculate the PPD for each target lesion and the SPD for all target lesions together. Response categories are as defined below:
Complete Response (CR): All target lymph nodes must have regressed to normal size defined as ≤1.5 cm in LDi; target extranodal sites must be absent (0 by 0 cm);
Partial Response (PR): ≥50% decrease in SPD of target lesions from baseline, and no individual lesion meets the criteria for progression;
Progressive Disease (PD): Target lesion progression is based on the progression of any single lesion (not a change in the SPD), which meets all of the following requirements:
   The lesion must have increased by ≥50% from its nadir in PPD;
   For a lymph node, it must be >1.5 cm in LDi, and for an extranodal lesion it must be ≥1.0 cm in LDi;
   And one of the following:
      For lesions ≤2 cm at nadir, the lesion's LDi or SDi must have increased by ≥0.5 cm at the current timepoint from its nadir; or
      For lesions >2 cm at nadir, the lesion's LDi or SDi must have increased by ≥1 cm at the current timepoint from its nadir;
Stable Disease (SD): A target lesion assessment of SD requires all of the following:
   Target lesions do not meet the criteria for CR or PR; and
   No individual lesion meets the criteria for progression;
Not Evaluable (NE): When a target lesion identified at baseline cannot be evaluated at a post-baseline timepoint due to missing imaging, poor image quality, changes in the lesion or background that precludes assessment, or focal therapy or other procedures that change the lesion size, the target lesion assessment will be NE, unless progression is assessed in another target lesion.

If two lesions merge, the entire resulting lesion is measured as a single target lesion, which will almost always result in progression, as lesions merge by growing. If a target lesion splits into fragments, each fragment is then followed as separate target lesions.

### Non-Target Lesions

Non-target lesions will be assessed at each post-baseline timepoint individually and as a group. Response categories are as defined below:
Absent/Normalized (CR): All individual non-target nodal lesions must have returned to normal size; all extranodal lesions must have disappeared;
Unequivocal Progression (PD): Any individual non-target lesion must unequivocally progress in the context of the overall disease burden to be assessed as PD; for increased thickening of the wall of a hollow viscus, the reviewer will use their cautious judgment to determine whether the increase is most likely caused by disease progression.

PD should not be called based on enlarging pleural effusions or ascites, or enlarging lytic bone lesions, and rather, the overall assessment should be based on the rest of the disease burden.
Stable Disease (SD): At least one non-target lesion is still present, or a node enlarged, without any individual lesions showing unequivocal progression;
Not Evaluable (NE): When an individual non-target lesion lymph node, extranodal lesion, or non-measurable disease cannot be assessed at a post-baseline timepoint due to missing imaging, poor image quality, changes in the lesion or background that precludes assessment, or focal therapy to a lesion, the individual non-target lesion will be assessed as NE; the assessment of the non-target lesions as a whole will be NE if any of the non-target lesions are NE and none are PD.

### New Lesions

Lesions will be considered new if they were not present at the baseline timepoint, but are visible at the current timepoint. A node consistent with lymphoma will be recorded as a new lesion if it was previously normal in size and is now >1.5 cm in LDi. An unequivocal, new extranodal lesion consistent with lymphoma of any size is considered a new lesion. If multiple new extranodal lesions are noted, at least one should be recorded as a new lesion.

New lesions must be consistent with lymphoma rather than another etiology (e.g., infection, inflammation) and must be PET positive, if PET is available. New lesions will be treated as PET positive when PET is not available to confirm avidity.

Some types of truly non-measurable lesions generally require further verification that they are attributable to lymphoma through biopsy or cytology. These include ascites, pleural or pericardial effusions, and lytic bone lesions. They may be recorded as new lesions only when there is other evidence of progression.

Other truly non-measurable lesions will not require verification to be considered a new lesion, as long as their appearance is unequivocal in the judgment of the reviewer:
Non-measurable lesions such as brain and CNS lesions including leptomeningeal disease attributable to lymphoma; and
Non-measurable nodal masses such as infiltrative mesenteric masses or retroperitoneal masses.

Extranodal lesions which disappeared and then reappeared at a later timepoint will have the same effect as a new lesion, but are not designated “new.”

### Spleen Response

The spleen will be measured in the craniocaudal length as at baseline, and the enlarged portion calculated by subtracting 13 cm. Response categories for the spleen are as defined below.
Normal (CR): Spleen was enlarged at baseline and has regressed to ≤13 cm at the current timepoint, or the spleen was assessed as normal at baseline and is still normal, or there is radiological evidence of splenectomy at baseline, or the spleen was normal at baseline, and there has been a splenectomy since then;
Partial Resolution (PR): Spleen was assessed as enlarged at baseline, and its excess length has decreased by ≥50%;
Stable Splenomegaly (SD): No decrease consistent with PR and no increase consistent with progression;
Unequivocal increase (PD): The spleen is assessed as PD if any of the following are true:
   Recurrent splenomegaly: A spleen which was abnormal at baseline (≥13 cm) first returned to normal, but at the current timepoint the spleen increases by >2 cm from its nadir and the length is >13 cm;
   New splenomegaly: No prior splenomegaly and spleen increases by >2 cm from baseline and the length is >13 cm; or
   Progression of existing splenomegaly: A spleen which is abnormal at baseline has the enlarged portion increase by >50%, and by ≥1 cm in absolute measurement, at the current timepoint from its nadir value.

### Liver Response

The liver will be assessed qualitatively. Response categories are as defined below:
Normal (CR): Liver was assessed as enlarged at baseline and has regressed to a normal size, or still enlarged but without evidence of lymphoma involvement, or the liver was assessed as normal at baseline and continues to be normal;
Stable Disease (SD): Liver is considered stable if there is persistent liver involvement with evidence based on imaging (CT or MRI) or biopsy that there is infiltration by lymphoma of the entire organ;
Unequivocal increase (PD): New hepatomegaly, recurrent hepatomegaly, or definite growth from prior liver size, and there is evidence that this is due to lymphoma infiltration.

### Anatomic Response

The anatomic response should be assessed at each post-baseline timepoint based on the criteria shown in Table 5 below. Liver size alone should never be the basis for response determination by itself, but only support the assessment as supplemental information.

TABLE 5
Anatomic Response Evaluation Criteria

| | | | | Anatomic |
|---|---|---|---|---|
| Target | Non-Target | Spleen | New Lesions | Response |
| CR | CR | CR | No | CR* |
| CR | CR | PR | No | PR** |
| CR | SD | CR | No | PR** |
| PR | CR/SD | CR/PR | No | PR |
| PR | CR/SD | SD | No | SD |
| SD | CR/SD | CR/PR | No | SD |
| SD | CR/SD | CR/PR/SD | No | SD |
| Any non-PD | NE | CR/PR/SD | No | NE |
| NE | Any non-PD | CR/PR/SD | No | NE |
| PD | Any | Any | Any | PD |
| Any | PD | Any | Any | PD |
| Any | Any | PD | Any | PD |
| Any | Any | Any | Yes | PD |

*If the liver shows lymphoma involvement, it must resolve to allow CR
**Spleen can drive PR on its own if no target lesions were identified at baseline

### 2.3.4 Metabolic Response

In addition to anatomic imaging, metabolic imaging using FDG-PET can contribute to the assessment, if it is available, or may form the sole basis for response if no anatomic imaging is performed at a time point. An FDG-PET is required at screening. Subsequent time points that require PET are shown in the schedule of activities (SoA). If lesions are not FDG-avid at baseline, PET is not required at follow-up timepoints unless clinically indicated.

### PET Assessment

For every FDG-PET scan, a 5PS score is obtained by comparing the maximum standardized uptake value (SUVmax) of the lesion that shows the greatest tracer uptake (the “hottest” lesion) to surrounding normal tissue, to a region of interest (ROI) placed over blood in the heart or major vessels of the mediastinum (the “mediastinal blood pool”) and to an ROI placed over normal liver. Depending on the uptake, a score between 1-5 will be assigned as follows:

TABLE 6
Pet 5PS Score

| Score | Definition |
|---|---|
| 1 | No uptake above background |
| 2 | Uptake above background, but below mediastinal blood pool |
| 3 | Uptake >mediastinal blood pool, but ≤uptake in liver |
| 4 | Uptake moderately higher than liver |
| 5 | Uptake markedly higher than liver (SUVmax > 2× |
| | normal liver) OR (after treatment has started) |
| | New FDG-positive lymphoma lesions |

After screening, in addition to the 5PS score the assessment of the FDG-PET also involves an assessment of the overall uptake (a combination of extent and intensity) by tissue consistent with lymphoma, and comparison of this uptake to the baseline and to the scan on which the overall uptake was lowest (nadir).

### Metabolic Response Determination

Metabolic response categories are defined as follows:

TABLE 7
Metabolic Response Categories

| Metabolic Response | Definition |
|---|---|
| Complete metabolic response | A score of 1, 2, or 3. |
| (CMR) | |
| Partial metabolic response | A score of 4 or 5 (without new lesions), |
| (PMR) | AND Overall uptake decreased compared to |
| | baseline |
| Stable metabolic disease | A score of 4 or 5 (without new lesions), AND |
| (SMD) | Overall uptake unchanged compared to baseline and nadir |
| Progressive metabolic | A score of 4 or 5 with overall uptake increased compared |
| disease (PMD) | to nadir, OR with new FDG-positive lesions consistent |
| | with lymphoma |

In Waldeyer's ring or extranodal sites with high physiologic uptake or with activation within spleen or marrow (e.g., with chemotherapy or myeloid colony-stimulating factors), uptake may be greater than normal mediastinum and/or liver. In this circumstance, CMR may be inferred if uptake at sites of initial involvement is no greater than surrounding normal tissue, even if the tissue has high physiologic uptake.

### 2.3.5 Clinical Data

### Bone Marrow Assessment

To allow an overall response of CR, the bone marrow must be clear of lymphoma (negative for lymphoma). Lugano allows assessment of bone marrow based on FDG-PET, if the lymphoma type is FDG-avid. Bone marrow on FDG-PET may be normal, may show diffuse uptake (clinical judgment is required, because this is also compatible with reactive changes due to chemotherapy or colony-stimulating factors), or may show focal increased uptake that is strongly indicative of lymphoma.

A negative PET allows the bone marrow to be declared negative, even without biopsy, and would support a CR overall (diffuse uptake compatible with reactive changes from chemotherapy or growth factor use can fall into this category). A PR may occur with residual uptake higher than uptake in normal marrow, but reduced compared with baseline. If there are persistent focal changes in the marrow in the context of a nodal response, consideration should be given to further evaluation with MRI or biopsy or an interval scan./

Bone marrow aspirate or biopsy is required only as clinically indicated, or if FDG-PET evaluation of bone marrow is judged inconclusive. For lymphoma that is shown at baseline to be non FDG-avid, aspiration or biopsy is required to declare marrow to be negative. If bone marrow biopsy is performed and shows lymphoma, the bone marrow is considered positive, regardless of the results of the PET.

### Physical Exam Findings

On rare occasions, lesions may be present on physical examination that are not seen on imaging at all. An example might be lymphadenopathy in the popliteal fossa, when the “whole body” imaging includes only anatomy to the mid-femur. Such lesions should be documented as non-target lesions in study forms. They can contribute to progression if new lesions appear this way, and if any were present at baseline, they must disappear for an overall response of CR.

### Other Clinical Data

Information on the use of hematopoietic growth factors and other medications can affect the response assessment as described above. At certain protocol-specified time points, additional tissue biopsies may be collected and incorporated into the response assessment.

### 2.3.6 Overall Response

Overall response at each timepoint is determined by combining the anatomic response, metabolic response, and clinical data. When both CT and FDG-PET are available, the overall response is driven primarily by the metabolic response. When only one imaging modality is available at a given timepoint, that modality is the main determinant of overall response.

TABLE 8
Overall Response Evaluation Criteria

| Metabolic | Anatomic | | | Overall |
|---|---|---|---|---|
| Response | Response | Bone Marrow | Physical Exam | Response |
| CMR | CR, PR, or SD | Negative | No lesions | CR |
| PMR | CR, PR, or SD | Any | No new lesions | PR |
| SMD | CR, PR, or SD | Any | No new lesions | SD |
| PMD | Any | Any | Any | PD |
| Any | PD | Any | Any | PD |

CMR = complete metabolic response; CR = complete response; PD = progressive disease; PMD = progressive metabolic disease; PMR = partial metabolic response; PR = partial response; SD = stable disease; SMD = stable metabolic disease.

During determination of overall response, if no FDG-PET was performed at the timepoint in question, the results of a preceding PET may be “carried forward”, unless there has been worsening of disease on the CT. For example, if a post-baseline assessment shows a CMIR on the PET, and PR on the CT, the overall response is CR. If the next timepoint shows continued PR on the CT, but there is no PET available, the overall response for that visit is still CR.

If there is partial response based on either anatomic or metabolic imaging, but a biopsy demonstrates that the tissue in question is not malignant, the response can be upgraded to a complete response.

### 2.4 Safety Assessments

The primary safety endpoints are DLTs, AEs and discontinuation of study treatment due to AEs. Safety and tolerability will be assessed by clinical review of all relevant parameters including AEs, laboratory tests, and vital signs. Safety parameters to be analyzed include, but are not limited to AEs, SAEs, fatal AEs, and laboratory changes. Laboratory samples to be obtained and parameters to be analyzed are indicated in Table 9.

TABLE 9
Safety Laboratory Assessments

Laboratory

| Assessments | Parameters | | |
|---|---|---|---|
| Hematology | Platelet Count | RBC Indices: | WBC count with |
| | RBC Count | MCV | Differential^{a}: |
| | Hemoglobin | MCH | Neutrophils |
| | Hematocrit | Reticulocytes | Lymphocytes |
| | | | Monocytes |
| | | | Eosinophils |
| | | | Basophils |

| | | | | |
|---|---|---|---|---|
| Chemistry | BUN^{b,e} | Potassium | AST/SGOT | Total bilirubin (and |
| | | | | direct bilirubin, if |
| | | | | total bilirubin is |
| | | | | above the ULN) |
| | Albumin | Bicarbonate^{e,f} | Chloride | Phosphorous |
| | Creatinine, CrCl^{c} or GFR | Sodium | ALT/SGPT | Total Protein |
| | Glucose (fasting or | Calcium | Alkaline | Magnesium |
| | nonfasting per | | phosphatase | |
| | institutional standard) | | | Uric Acid |
| | LDH | Amylase | Lipase | Creatine Kinase |

| | |
|---|---|
| Routine | Specific gravity |
| Urinalysis/Urine | pH, glucose, protein, blood, ketones, nitrite, leukocyte esterase |
| Dipstick Testing | Microscopic examination (if blood or protein is abnormal) |
| Pregnancy Testing | Highly sensitive serum or urine hCG pregnancy test (as needed for WOCBP) |
| | Note: see country-specific requirements in Appendix 7 |
| Other Screening | Coagulation: PT/INR and aPTT/PTT^{d} |
| Tests | FSH (as needed in WONCBP only) |
| | Serology (HIV antibody, hepatitis B surface antigen [HBsAg], and hepatitis C |
| | virus antibody) as required by local health authority or institutional regulations. |
| | Refer to Appendix 7 for country-specific information. |
| Other Tests | Serum Beta 2-microglobulin |

ALT = alanine aminotransferase; aPTT-activated partial thromboplastin time; AST=aspartate aminotransferase; BUN = blood urea nitrogen; CrCl = creatinine clearance; FSH = follicle-stimulating hormone; GFR = glomerular filtration rate; hCG=human chorionic gonadotropin; INR = international normalized ratio; LDH = lactate dehydrogenase; MCH = mean corpuscular hemoglobin; MCV = mean corpuscular volume; PT = prothrombin time; PTT = partial thromboplastin time; RBC = red blood cell; SGOT = serum glutamic oxaloacetic transaminase; SGPT = serum glutamic-pyruvic transaminase; ULN = upper limit of normal; WBC = white blood cell; WOCBP = women of childbearing potential; WONCBP = women of nonchildbearing potential
^{a}Absolute or % acceptable per institutional standard.
^{b}Blood urea nitrogen is preferred, but if not available, then urea may be tested. Record either BUN or urea result, but not both.
^{c}Creatinine clearance should be calculated using Cockcroft-Gault.
$\begin{matrix}\underline{[ [ 140 - age ( years ) ] \times weight ( kg ) ]} {( \times F )}^{*} \\ Serum creatinine ( mg / dL ) \times 72\end{matrix}$ *where F = 0.85 for females and F = 1 for males
^{d}Coagulation tests must include PT or INR plus aPTT or PTT
^{e}If the local laboratory is unable to perform these tests, the site should submit the sample to the central laboratory for testing.
^{f}Bicarbonate is preferred; if not available, Carbon Dioxide may be tested

### 2.4.1 AE Definition

Adverse events will be monitored throughout the study and graded in severity according to the guidelines outlined in the NCJ CTCAE Version 5.0. An AL is any untoward medical occurrence in a clinical study participant, temporally associated with the use of study intervention, whether or not considered related to the study intervention. An AL can therefore be any unfavorable and unintended sign (including an abnormal laboratory finding), symptom, or disease (new or exacerbated) temporally associated with the use of a study intervention.

For purposes of AE definition, study intervention includes any pharmaceutical product, biological product, vaccine, diagnostic agent, medical device, combination product, or protocol specified procedure whether investigational or marketed (including placebo, active comparator product, or run-in intervention), manufactured by, licensed by, provided by, or distributed by the Sponsor for human use in this study.

### Events Meeting the AE Definition

Any abnormal laboratory test results (hematology, clinical chemistry, or urinalysis) or other safety assessments (e.g., ECG, radiological scans, vital signs measurements), including those that worsen from baseline, considered clinically significant in the medical and scientific judgment of the investigator;
Exacerbation of a chronic or intermittent preexisting condition including either an increase in frequency and/or intensity of the condition;
New conditions detected or diagnosed after study intervention administration even though it may have been present before the start of the study;
Signs, symptoms, or the clinical sequelae of a suspected drug-drug interaction;
Signs, symptoms, or the clinical sequelae of a suspected overdose of either study intervention or a concomitant medication; and
For all reports of overdose (whether accidental or intentional) with an associated AE, the AE term should reflect the clinical symptoms or abnormal test result; an overdose without any associated clinical symptoms or abnormal laboratory results is reported using the terminology “accidental or intentional overdose without adverse effect.”

### Events NOT Meeting the AE Definition

Medical or surgical procedure (e.g., endoscopy, appendectomy): the condition that leads to the procedure is the AE;
Situations in which an untoward medical occurrence did not occur (social and/or convenience admission to a hospital);
Anticipated day-to-day fluctuations of preexisting disease(s) or condition(s) present or detected at the start of the study that do not worsen; and
Surgical procedure(s) planned prior to informed consent to treat a preexisting condition that has not worsened.

### 2.4.2 Definition of SAE

If an event is not an AE per definition above, then it cannot be a serious adverse event even if serious conditions are met. An SAE is defined as any untoward medical occurrence that, at any dose:
Results in death;
Is life-threatening;
   The term “life-threatening” in the definition of “serious” refers to an event in which the participant was at risk of death at the time of the event; it does not refer to an event, which hypothetically might have caused death, if it were more severe;
Requires inpatient hospitalization or prolongation of existing hospitalization;
   Hospitalization is defined as an inpatient admission, regardless of length of stay, even if the hospitalization is a precautionary measure for continued observation;
   Hospitalization for an elective procedure to treat a preexisting condition that has not worsened is not an SAE; preexisting condition is a clinical condition that is diagnosed prior to the use of an MSD product and is documented in the participant's medical history.
Results in persistent or significant disability/incapacity;
   The term disability means a substantial disruption of a person's ability to conduct normal life functions; this definition is not intended to include experiences of relatively minor medical significance such as uncomplicated headache, nausea, vomiting, diarrhea, influenza, and accidental trauma (e.g., sprained ankle) that may interfere with or prevent everyday life functions but do not constitute a substantial disruption;
Results in a congenital anomaly/birth defect;
   In offspring of participant taking the product regardless of time to diagnosis;
Other important medical events;
   Medical or scientific judgment should be exercised in deciding whether SAE reporting is appropriate in other situations such as important medical events that may not be immediately life-threatening or result in death or hospitalization but may jeopardize the participant or may require medical or surgical intervention to prevent 1 of the other outcomes listed in the above definition; these events should usually be considered serious;
   Examples of such events include invasive or malignant cancers, intensive treatment in an emergency room or at home for allergic bronchospasm, blood dyscrasias or convulsions that do not result in hospitalization, or development of drug dependency or drug abuse.

### 2.4.3 Assessment of Intensity/Toxicity

An event is defined as “serious” when it meets at least 1 of the predefined outcomes as described in the definition of an SAE, not when it is rated as severe. The investigator will make an assessment of intensity for each AE and SAE (and other reportable safety event) according to the 10 NCI CTCAE, version 5. Any AE that changes CTCAE grade over the course of a given episode will have each change of grade recorded on the AE CRFs/worksheets.
Grade 1: Mild; asymptomatic or mild symptoms; clinical or diagnostic observations only; intervention not indicated;
Grade 2: Moderate; minimal, local or noninvasive intervention indicated; limiting age-appropriate instrumental ADL;
Grade 3: Severe or medically significant but not immediately life-threatening; hospitalization or prolongation of hospitalization indicated; disabling; limiting self-care ADL;
Grade 4: Life threatening consequences; urgent intervention indicated; or
Grade 5: Death related to AE.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention.

1. A method of treating a B-cell malignancy, comprising co-administering to a subject in need thereof an immunoconjugate of Formula (I), 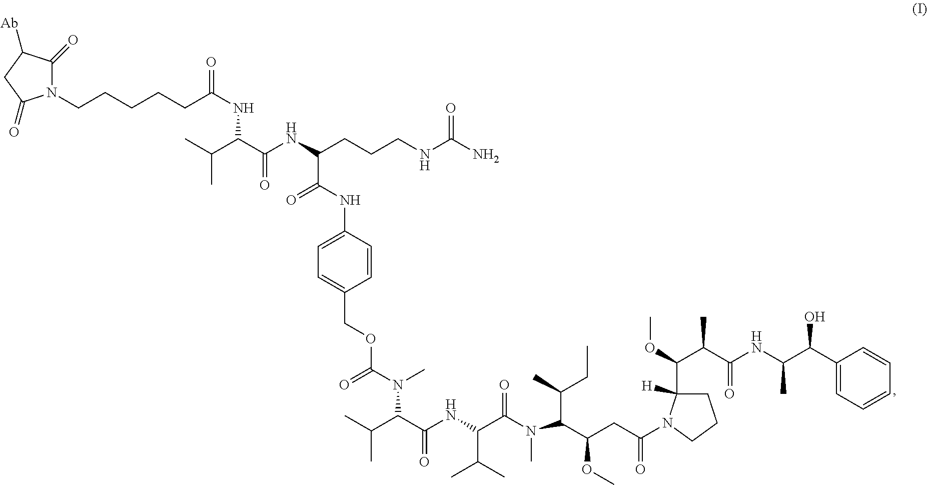
and a compound of Formula (II), or a pharmaceutically acceptable salt thereof, 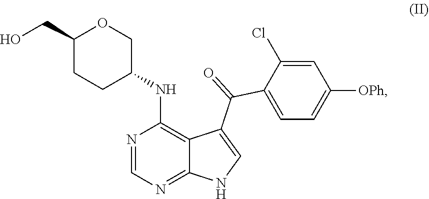 [SMILES: O=C(c1ccc(Oc2ccccc2)cc1Cl)c1c[nH]c2ncnc(N[C@@H]3CC[C@@H](CO)OC3)c12]
wherein the immunoconjugate comprises an antibody (Ab) conjugated to a drug moiety shown in Formula (I), and wherein the antibody comprises a heavy and light chain comprising the amino acid sequences of SEQ ID NOs: 1 and 2, respectively.

2. The method of claim 1, wherein the number of the drug moiety per antibody (DAR) is from 1 to 7.

3. (canceled)

4. The method of claim 1, wherein the number of the DAR is from 3 to 5.

5. The method of claim 1, wherein the immunoconjugate of Formula (I) is administered to the subject at a dose from 0.25 to 4.00 mg/kg.

6. The method of claim 1, wherein the immunoconjugate of Formula (I) is administered to the subject at a dose of 2.00, 2.25, or 2.50 mg/kg.

7. The method of claim 1, wherein the compound of Formula (II) is administered to the subject at a dose of 5 to 75 mg per day.

8. (canceled)

9. The method of claim 1, wherein the immunoconjugate of Formula (I) is administered in three-week cycles and administered on Day 1 of each cycle at said dose.

10. The method of claim 1, wherein the immunoconjugate of Formula (I) is administered in three-week cycles and administered on Days 1 and 8 of each cycle at said dose.

11. The method of claim 1, wherein the immunoconjugate of Formula (I) is administered in four-week cycles and administered on Days 1, 8, and 15 of each cycle at said dose.

12. The method of claim 9, wherein the number of cycles is 2 or more.

13. (canceled)

14. The method of claim 1, wherein the compound of Formula (II) is administered one, two, three, or four times per day.

15. (canceled)

16. The method of claim 1, wherein the compound of Formula (II) is administered:
La) every day for at least one day,
(b) every day for at least one week,
(c) every day for at least three weeks,
(d) every day for at least three months, or
(e) every day for at least twelve months.

17-23. (canceled)

24. The method of claim 1, wherein the B-cell malignancy is a mantle cell lymphoma (MCL).

25. The method of claim 1, wherein the patient has been treated previously for the B-cell malignancy.

26. (canceled)

27. The method of claim 1, wherein co-administration of the immunoconjugate of Formula (I) and compound of Formula (II) results in one or more of the following:
a) disease regression;
b) delayed disease progression;
c) prevention of disease recurrence or residual disease;
d) decrease of the size of nodal or extranodal malignant masses;
e) decrease of the malignant cell numbers in bone marrow and/or peripheral blood;
f) decrease of malignant splenomegaly or hepatomegaly;
g) improved anemia, neutropenia, or thrombocytopenia;
h) ameliorated cutaneous manifestation;
i) ameliorated disabling constitutional symptoms;
j) prolonged survival; and
k) complete disease eradication.

28. (canceled)

29. The method of claim 1, wherein the immunoconjugate of Formula (I) is zilovertamab vedotin.

30. The method of claim 1, wherein the compound of Formula (II) is nemtabrutinib.

31-36. (canceled)

37. A method of treating a B-cell malignancy, comprising co-administering to a subject in need thereof an immunoconjugate of Formula (I), 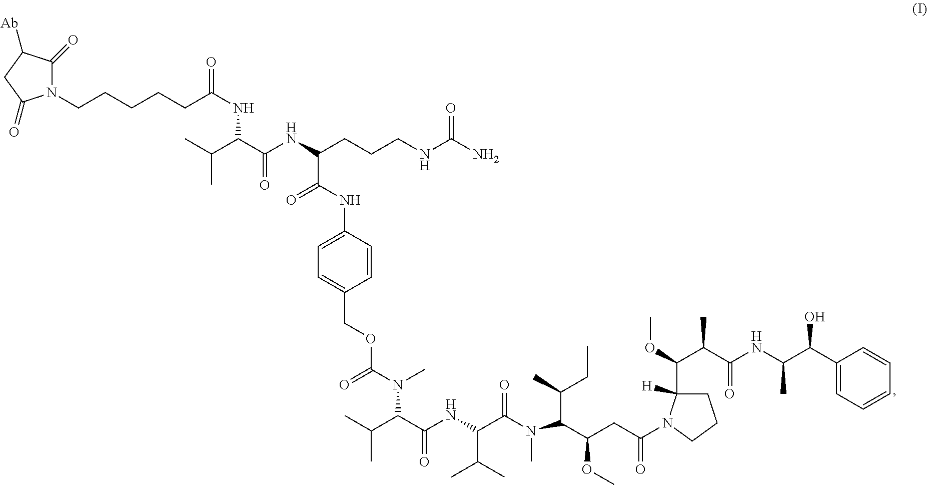
and a compound of Formula (II), or a pharmaceutically acceptable salt thereof, 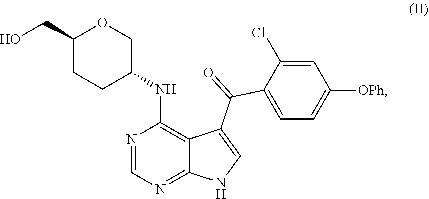 [SMILES: O=C(c1ccc(Oc2ccccc2)cc1Cl)c1c[nH]c2ncnc(N[C@@H]3CC[C@@H](CO)OC3)c12]
wherein the immunoconjugate comprises an antibody (Ab) conjugated to a drug moiety shown in Formula (I), wherein the number of the drug moiety per antibody (DAR) ranges from 3 to 5, wherein the antibody comprises a heavy and light chain comprising the amino acid sequences of SEQ ID NOs: 1 and 2, respectively, wherein the immunoconjugate of Formula (I) is administered to the subject in three-week cycles at a dose of 2.00, 2.25, or 2.50 mg/kg on Days 1 and 8 of each cycle, wherein the compound of Formula (II) is administered to the subject at a dose of 65 mg daily, and wherein the B-cell malignancy is a mantle cell lymphoma (MCL).

38. The method of claim 37, wherein the immunoconjugate of Formula (I) is zilovertamab vedotin.

39. The method of claim 37, wherein the compound of Formula (II) is nemtabrutinib.

40. A pharmaceutical composition comprising effective amounts of an immunoconjugate of Formula (I), 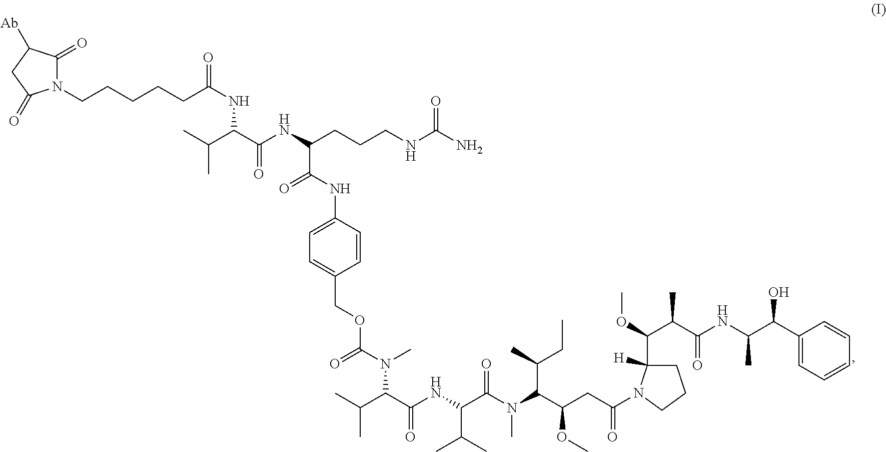
and a compound of Formula (II), or a pharmaceutically acceptable salt thereof, 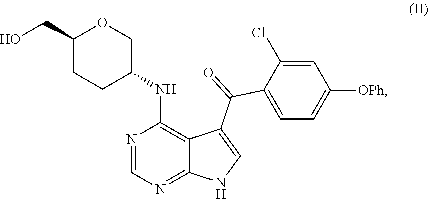 [SMILES: O=C(c1ccc(Oc2ccccc2)cc1Cl)c1c[nH]c2ncnc(N[C@@H]3CC[C@@H](CO)OC3)c12]
wherein the immunoconjugate comprises an antibody (Ab) conjugated to a drug moiety shown in Formula (I), wherein the number of the drug moiety per antibody (DAR) ranges from 3 to 5, wherein the antibody comprises a heavy and light chain comprising the amino acid sequences of SEQ ID NOs: 1 and 2, respectively; and a pharmaceutically acceptable carrier.

41. The pharmaceutical composition of claim 40, wherein the immunoconjugate of Formula (I) is zilovertamab vedotin.

42. The pharmaceutical composition of claim 40, wherein the compound of Formula (II) is nemtabrutinib.

43. The pharmaceutical composition of claim 41, wherein the effective amount of zilovertamab vedotin is a dose of 2.00, 2.25, or 2.50 mg/kg.

44. The pharmaceutical composition of claim 42, wherein the effective amount of nemtabrutinib is a dose of 65 mg.

45. (canceled)


SEQUENCE LISTING (10 sequences)

SEQ ID NO: 1 - protein, 446 aa, synthetic construct
QVQLQESGPGLVKPSQTLSLTCTVSGYAFTAYNIHWVRQAPGQGLEWMGSFDPYDGGSSYNQKFKDRLTISKDTSKNQVVLTMTNMDPVDTATYYCARGWYYFDYWGHGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 214 aa, synthetic construct
DIVMTQTPLSLPVTPGEPASISCRASKSISKYLAWYQQKPGQAPRLLIYSGSTLQSGIPPRFSGSGYGTDFTLTINNIESEDAAYYFCQQHDESPYTFGEGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 116 aa, synthetic construct
QVQLQESGPGLVKPSQTLSLTCTVSGYAFTAYNIHWVRQAPGQGLEWMGSFDPYDGGSSYNQKFKDRLTISKDTSKNQVVLTMTNMDPVDTATYYCARGWYYFDYWGHGTLVTVSS

SEQ ID NO: 4 - protein, 107 aa, synthetic construct
DIVMTQTPLSLPVTPGEPASISCRASKSISKYLAWYQQKPGQAPRLLIYSGSTLQSGIPPRFSGSGYGTDFTLTINNIESEDAAYYFCQQHDESPYTFGEGTKVEIK

SEQ ID NO: 5 - protein, 8 aa, synthetic construct
GYAFTAYN

SEQ ID NO: 6 - protein, 8 aa, synthetic construct
FDPYDGGS

SEQ ID NO: 7 - protein, 7 aa, synthetic construct
GWYYFDY

SEQ ID NO: 8 - protein, 6 aa, synthetic construct
KSISKY

SEQ ID NO: 9 - skipped

SEQ ID NO: 10 - protein, 8 aa, synthetic construct
QQHDESPY